(19) United States
(12) Patent Application Publication
(10) Pub. No.: US 2006/0229239 A9
(43) Pub. Date: Oct. 12, 2006

(54) Novel curcuminoid-factor VIIa constructs as suppressors of tumor growth and angiogenesis

(76) Inventors: Mamoru Shoji, Conyers, GA (US); James Snyder, Atlanta, GA (US); Dennis Liotta, Atlanta, GA (US); Aiming Sun, Atlanta, GA (US)

Correspondence Address: PATREA L. PABST;PABST PATENT GROUP LLP, 400 COLONY SQUARE, SUITE 1200, ATLANTA, GA, 30361, US

(21) Appl. No.: 10/383,898

(22) Filed: Mar. 7, 2003

Related U.S. Application Data
(60) Provisional application No. 60/362,762, filed on Mar. 8, 2002.
(60) Provisional application No. 60/403,794, filed on Aug. 14, 2002.

(51) Int. Cl. A61K 38/17

(52) U.S. Cl. 514/12

(57) ABSTRACT

The fluorinated curcuminoid (3,5-bis-(2-fluorobenzylidene)-piperidin-4-one-acetate is about ten times more effective at arresting the growth of tumor cells than cisplatin. The present invention provides methods to deliver a cytotoxic compound, such as a curcuminoid, specifically to cancer cells and to the vascular endothelial cells that nourish solid tumors. The method involves tethering the drug to a protein such as in factor VIIa that retains high affinity for the surface protein tissue factor. Upon complexation, the resulting heterodimer is endocytosed and the drug is subsequently liberated inside the target cell via proteolytic cleavage. The present invention further provides for the synthesis of novel curcuminoid-tether-linker-factor VIIa compositions and for methods of delivery of effective doses of the novel compositions to target tumor or endothelial cells in a patient

The present application claims the benefit of priority from a provisional application filed Mar. 8, 2002 and having U.S. Serial No. 60/362,762.

### ACKNOWLEDGMENT OF FEDERAL RESEARCH SUPPORT

This invention was made, at least in part, with funding from the National Institutes of Health with grant 1 R21 CA82995-01A1 and Department of Defense, Department of U.S. Army grant DAMD17-00-1-0241. Accordingly, the United States Government has certain rights in this invention.

### FIELD OF THE INVENTION

The present invention relates to novel compositions for selectively delivering a curcuminoid to a target cell. The present invention further relates to methods for synthesizing said novel compositions and for delivering them to tissue factor-bearing target cells.

### BACKGROUND

The association between malignant disease and the hypercoagulable state was documented more than 100 years ago. A critical role for tumor-derived vasoactive factors like vascular endothelial growth factor (VEGF) in the formation of the blood vessels that nourish tumors has been emphasized in more recent work. Cell-associated procoagulants like tissue factor (TF) have also been implicated in the pathogenesis of these events. “Tissue factor” is a transmembrane protein receptor specific for coagulation factor VII (and its activated form factor VIIa (fVIIa)), and is the primary regulator of blood coagulation. When bound to the extracellular domain of TF, fVIIa activates factor X (fX) via the extrinsic pathway. Alternatively, TF-VIIa indirectly activates fX via the activation of factor IX in the intrinsic blood clotting pathway. Independent of the potent procoagulant function, TF may act as a modulator of VEGF expression and as a cell signal transducer. These studies have provided important evidence for a dynamic interaction between host inflammatory cells, tumor cells and vascular endothelial cells (VECs). “Leaky blood vessels,” perfusion of tumors with fibrinogen and conversion of the fibrinogen to fibrin by cell-associated procoagulants in the local tumor microenvironment are some of the consequences. These events may occur at the blood vessel wall during hematogenous spread of tumors or within the extravascular space as primary tumors or metastasis grow. Fibrin may be generated by the expression of procoagulant activity, particularly tissue factor expressed on the surface of tumor cells, tumor-associated macrophages and tumor-associated VECs.

Increased tumor angiogenesis is associated with a poor prognosis in a variety of human tumors, including invasive breast cancer, early stage and node negative breast cancer, prostate carcinoma and adenocarcinoma of the lung. There is a statistically significant correlation between so-called tumor microvascular density and relapse-free survival. It has been shown that tumor cells secrete a number of angiogenic factors, including VEGF, interleukin-8 (IL-8) and basic fibroblast growth factor (bFGF), and endothelial cell proliferation is faster in tumors compared with normal tissues.

Tumor cells secrete factors that increase vessel permeability. Vascular permeability factor, or VEGF, purified originally from tumor cells has a molecular weight of 45 kDa and acts specifically on VECs to promote vascular permeability, endothelial cell growth and angiogenesis. VEGF induces expression of TF activity in VECs and monocytes and is chemotactic for monocytes, osteoblasts and VECs. VEGF promotes extravasation of plasma fibrinogen, which can be converted to fibrin by TF-dependent mechanisms. Fibrin deposition alters the tumor extracellular matrix to promote the migration of macrophages, fibroblasts and endothelial cells.

Overexpression of the TF gene in murine tumor cells leads to increased VEGF and decreased transcription of thrombospondin (TSP), an endogenous antiangiogenic factor. When grown in immunodeficient mice, the TF-producing cells stimulate angiogenesis by approximately 2-fold, whereas low TF producers inhibit angiogenesis. This effect of TF is independent of its clot-promoting activated procoagulation activity. Human melanoma cells, transfected to hyperexpress TF, demonstrate greater metastatic potential than those with low TF expression. This pro-metastatic effect of TF requires the procoagulant function of the extracellular domain of TF and its cytoplasmic domain. Tissue Factor, therefore, regulates angiogenic properties of tumor cells by regulating the production of growth regulatory molecules that can act on VECs. There is also a critical role for TF expression in blood vessel development in both mice and human embryos. TF appears to have the dual function of regulating angiogenesis and vasculogenesis.

Malignant human breast cancers and melanomas express high levels of TF and VEGF. TF is also expressed on the surface of vascular endothelial cells (VECs) within the tumor micro environment of invasive breast cancer and adenocarcinoma of the lung. There is a strong relationship between the synthesis of TF and VEGF levels in human breast cancer cell lines and in human melanoma cell lines, and there is co-localization of TF- and VEGF-specific mRNAs.

The signal for VEGF synthesis in cancer cells is mediated via serine residues of the TF cytoplasmic tail which contains two serine residues that can be substrates for protein kinase C. Expression of TF and VEGF in cancer cells is further enhanced under hypoxic condition, and TF may function as a growth factor receptor. Factor VIIa may induce cell signaling via PKC-dependent phosphorylation, mitogen-activated protein kinase (MAPK) pathways and subsequently, via the transcription factors NF-κB and AP-1.

Curcumin, a yellow-colored spice used in curry and a product of turmeric, inhibits tumor necrosis factor- and phorbol ester-induced TF synthesis in VECs by blocking the transcription factors NF-κB, AP-1 and Egr-1. Curcumin can also inhibit TF and VEGF synthesis of human melanoma cell lines and prostate cancer cell lines, as well as bFGF-induced angiogenesis.

What is needed is a method for coupling curcumin to factor VIIa, the specific ligand for TF, while maintaining the affinity of the coupled-VIIa for TF.

What also is needed are methods for delivering curcumin and curcumin derivatives (curcuminoids) to the specific target, i.e., TF, which is aberrantly expressed on tumor cells and vascular endothelial cells in the tumor micro-environment. Inhibition of TF synthesis will block VEGF synthesis and tumor angiogenesis. What is also needed, therefore, is a method for coupling curcuminoids to active-site inactivated factor VIIa, the specific ligand for TF, while maintaining the affinity of the coupled-VIIa for TF.

### SUMMARY OF THE INVENTION

One aspect of the present invention provides novel compositions comprising cytotoxic compounds such as synthetic antitumor and anti-angiogenesis curcumin analogs (curcuminoids) linked to a protein delivery vehicle that can deliver the cytotoxic compoun specifically to cancer cells and vascular endothelial cells having surface-bound tissue factor. Novel compositions of the present invention can comprise a curcuminoid covalently linked to a tether which may be, but is not limited to, a dicarboxylic acid such as succinate. The tether is covalently linked to a N-terminal amino acid of a peptidyl linker such as phenylalanine-phenylalanine-arginine, the C-terminal amino acid of which comprises a methylketone. The methylketone group forms a covalent bond with an amino acid side group of factor VIIa (fVIIa) that does not prevent the conjugated construct from selectively binding to tissue factor expressed on a cell membrane. Preferably, the curcuminoid-tether-linker will have bonded to an amino acid of the serine protease domain of the fVIIa, thereby blocking the procoagulating activity of the novel therapeutic composition. The present invention also provides methods of synthesis of cytotoxic compound-protein conjugates. The compositions and methods of the present invention may increase the efficacy of the cytotoxic agents and decrease their side effects by delivering the agents to specific target cells. One of the curcumin analogs, EF24, that is useful in the present invention, was about 10 times more potent than cisplatin, which is a well-known anticancer agent currently in clinical use. The conjugate EF24-FFRck-fVIIa construct of the present invention kills cancer cell lines and vascular endothelial cells, such as HUVECs, that express tissue factor on the cell surface. The conjugate does not kill normal cells that do not express tissue factor. EF24-FFRck that is not coupled to fVIIa does not kill either cancer cells or normal cells regardless of the presence or absence of tissue factor expression on the cell surface because it cannot bind to any cells. Unconjugated EF24 alone indiscriminatingly kills normal cells, as well as cancer cells, irrespective of the level of tissue factor expression on the cell surface.

The methods of the present invention are particularly useful for delivering a drug to the blood vessels that feed cancer cells, thereby interrupting the supply of nutrients and oxygen and starving cancer cells. The methods are also useful for overcoming shortcomings of current cancer gene therapies that are unable to deliver drugs or genes intravenously because most of cancers and their metastatic foci are inaccessible by a direct injection.

The technology of the present invention will be able to deliver therapeutic agents only to cancer cells, vascular endothelial cells in a tumor and metastatic foci anywhere in the body intravenously, intraperitoneally, subcutaneously, and intra-tumoraly, providing the target cells express surface bound tissue factor. The analogs are also coupled to fVIIa so as to inactivate the active site of fVIIa so that besides acting as anticancer agents the curcminoid-conjugated inactivated fVIIa may also inhibit blood clotting by competing with native fVIIa. This will be therapeutic advantage for cancer patients since many such patients experience blood clotting problems due to cancer cells that express tissue factor escaping into the circulation and triggering blood coagulation.

The compositions and methods of the present invention are useful for treating any disease that requires targeted delivery of antiangiogenesis therapy including, but not limited to, reocclusion of the coronary artery. Restenosis will occur in 50% of angioplasty cases leading to myocardial infarction or angina pectoris. In angioplasty, the inner most layer of a treated blood vessel (vascular endothelial cells) is denuded. Tissue factor is then expressed on the exposed smooth muscle layer which proliferates and often re-obstructs the coronary artery. The methods of the present invention, therefore, are useful for delivering a drug specifically to the vascular smooth muscle cells that express tissue factor so as to inhibit the cell proliferation.

Other pathological conditions that may be regulated using the compositions and methods of the present invention include, but are not limited to, diabetic retinopathy that also involves the uncontrollable growth of blood vessels, expressing tissue factor, in the retina and leads to blindness in diabetic patients. Brain infarction results from blood clots triggered by atherosclerosis and vasculitis where tissue factor is likely to be expressed. Blood vessels of early lesions of rheumatoid arthritis also express tissue factor.

One aspect of the present invention, therefore, provides pharmaceutically acceptable compositions which comprise a therapeutically-effective amount of a cytotoxic composition-protein conjugate together with one or more pharmaceutically acceptable carriers (additives) and/or diluents for administering to an animal or human patient. The preferred route of administration is intravascular injection so that the effective dose of the curcuminoid can be delivered to a tumor via the vascular system. The dose may be delivered by subcutaneous injection, intraperitoneal injection, direct injection into the tumor or a proximal blood vessel feeding the tumor for reducing dilution of the effective therapeutic composition, and to achieve more rapid application of the composition to the tissue factor-bearing target tumor and/or vascular cells. The affinity of the fVIIa carrier polypeptide for tissue factor will localize the effective dose of the therapeutic composition for selectively targeting proliferating tumor and endothelial cells contributing to neovascularization of a tumor and to prevent metastasis of the tumor cells themselves.

Another aspect of the present invention, therefore, is the regulation of tissue factor and vascular endothelial growth factor by curcumin derivatives delivered to human and animal cells by factor VIIa. The present invention provides compositions and methods for delivering curcumin and curcumin derivatives to the specific target, i.e., tissue factor, which is aberrantly expressed on tumor cells and vascular endothelial cells in the tumor micro-environment. Inhibition of tissue factor synthesis will block VEGF synthesis and tumor anglogenesis.

### BRIEF DESCRIPTION OF THE FIGURES

FIG. 1 illustrates the amino acid sequence SEQ ID NO:1 of factor VII (fVIIa). Letters in bold indicate the cleavage point for conversion of the single-chain fVII to two-chain fVIIa, and the His193 that receives a covalently bonded arginyl-chloromethyl ketone of a peptidyl linker.

FIG. 2 illustrates the mass shift of fVIIa when modified by the covalent attachment of EF24-tether-linkers.

FIG. 3 illustrates the mean growth inhibitory concentrations of various curcuminoids when added to cultures of immortalized endothelial cells.

FIG. 4 illustrates mean growth inhibitory concentrations of various curcuminoids when tested against a panel of cultured tumor cells.

FIG. 5 illustrates the mean growth inhibitory concentrations of various curcuminoids when added to cultures breast cancer cells.

### DETAILED DESCRIPTION OF THE INVENTION

Reference now will be made in detail to the presently preferred embodiments of the invention. It will be apparent to those skilled in the art that various modifications, combinations, additions, deletions and variations can be made in the present invention without departing from the scope or spirit of the invention. For instance, features illustrated or described as part of one embodiment can be used in another embodiment to yield a still further embodiment. It is intended that the present invention cover such modifications, combinations, additions, deletions and variations as fall within the scope of the appended claims and their equivalents.

Throughout this application various publications are referenced. The disclosures of these publications are hereby incorporated by reference in their entireties in this application to more fully describe the state of the art to which this invention pertains.

For convenience, certain terms employed in the specification, examples, and appended claims are collected here.

Definitions

As used in this specification and the appended claims, the singular forms “a,” “an” and “the” include plural references unless the content clearly dictates otherwise. Thus, for example, reference to “a carrier” includes a mixture of two or more carriers.

As used herein the terms “polypeptide” and “protein” refer to a polymer of amino acids of three or more amino acids in a serial array, linked through peptide bonds. The term “polypeptide” includes proteins, protein fragments, protein analogues, oligopeptides and the like. The term “polypeptides” also contemplates polypeptides as defined above that are encoded by nucleic acids, produced through recombinant technology, isolated from an appropriate source, or are synthesized. The term “polypeptide” further contemplates polypeptides as defined above that include chemically modified amino acids or amino acids covalently or noncovalently linked to labeling ligands.

The term “truncated” as used herein refers to a polypeptide or protein that has less amino acids than a parent polypeptide or protein. It is contemplated that the difference in the amino acid sequence may be at one or both of the termini of an amino acid sequence or due to amino acids deleted from the interior of the sequence when compared to the parent amino acid sequence.

The term “linker” as used herein refers a molecule capable of covalently connecting a cytotoxic compound to an amino acid side chain of a protein. The term “linker” may be a non-peptidyl linker or a peptidyl linker. The linker may optionally have covalently bonded thereto a tether, as defined below, for covalently linking a cytotoxic compound to the linker. The term “peptidyl linker” as used herein refers to a peptide comprising at least two amino acids and which can be coupled to an amino acid side-chain of a protein. The linker may have a reactive group at the carboxyl terminus such as, but not limited to, a chloromethylketone. The peptide of the peptidyl linker may be cleavable by proteolytic enzymes found within a cell.

The term “tether” as used herein refers to a molecule that can form a hydrolysable bond such as, but not limited to, a carbamate, an amide, an ester, a carbonate or a sulfonate bond with a cytotoxic compound such as, but not limited to, a curcuminoid, and which can also be covalently bonded to a linker such as, but not limited to, the N-terminus of a linker, including a peptidyl linker, thereby connecting the cytotoxic compound to the linker. Suitable tethers for use in the present invention include, but are not limited to, a dicarboxylic acid, a disulfonic acid, an omega-amino carboxylic acid, an omega-amino sulfonic acid, an omega-amino carboxysulfonic acid, or a derivative thereof, wherein the tether may comprise 2-6 carbons in any arrangement such as a linear, branched or cyclic carbon arrangement, and wherein the tether is capable of forming a hydrolysable bond.

The term “cytotoxic compound” as used herein refers to a compound that, when delivered to a cell, either to the interior of a target cell or to the cell surface, is capable of killing the cell or otherwise inhibiting the proliferation of the target cell. The cytotoxic compound can be any such molecule that can form an amide or ester bond or otherwise be covalently bonded to a tether or a peptidyl linker and thereby connected to a protein that can selectively bind to a surface marker of a cell.

The terms “cell surface antigen” and “cell surface marker” as used herein may be any antigenic structure on the surface of a cell. The cell surface antigen may be, but is not limited to, a tumor associated antigen, a growth factor receptor, a viral-encoded surface-expressed antigen, an antigen encoded by an oncogene product, a surface epitope, a membrane protein which mediates a classical or atypical multi-drug resistance, an antigen which mediates a tumorigenic phenotype, an antigen which mediates a metastatic phenotype, an antigen which suppresses a tumorigenic phenotype, an antigen which suppresses a metastatic phenotype, an antigen which is recognized by a specific immunological effector cell such as a T-cell, and an antigen that is recognized by a non-specific immunological effector cell such as a macrophage cell or a natural killer cell. Examples of “cell surface antigens” within the scope of the present invention include, but are not limited to, CD5, CD30, CD34, CD45RO, CDw65, CD90 (Thy-1) antigen, CD117, CD38, and HLA-DR, AC133 defining a subset of CD34⁺ cells, CD19, CD20, CD24, CD10, CD13, CD33 and HLA-DR. Also contemplated to be within the scope of the present invention are cell surface molecules, including carbohydrates, proteins, lipoproteins or any other molecules or combinations thereof, that may be detected by selectively binding to a ligand or labeled molecule by methods such as, but not limited to, flow cytometry, FRIM, fluoresence microscopy and immunohistochemistry.

The term “tissue factor” as used herein refers to a transmembrane protein receptor for coagulation factor VII (and the activated form factor VIIa (fVIIa)), and is the primary regulator of blood coagulation.

The term “fVII” means “single chain” coagulation factor VII that may have the amino acid sequence SEQ ID NO: 1, or a trucncated or modified form thereof.

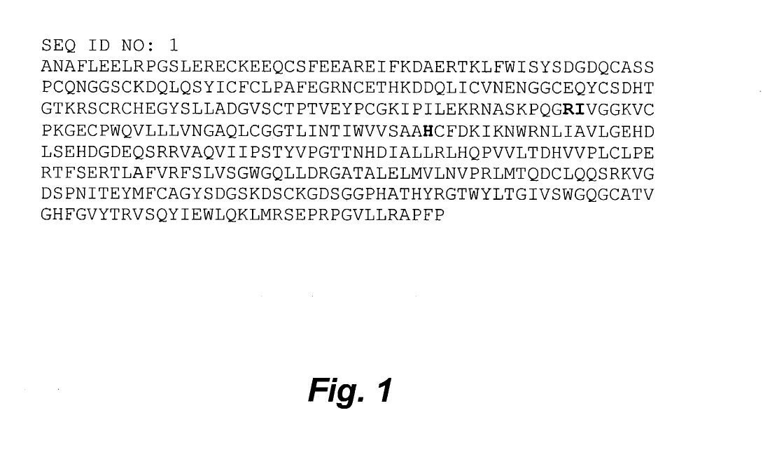

The term “factor VIIa”, or “fVIIa” means “two chain” activated coagulation factor VII cleaved by specific cleavage at the Arg152-Ile153 peptide bond. The uncleaved factor VII has the contiguous sequence as illustrated in FIG. 1. Factor VIIa, may be purified from blood or produced by recombinant means. It is evident that the practice of the methods described herein is independent of how the purified factor VIIa is derived and, therefore, the present invention is contemplated to cover use of any factor VIIa preparation suitable for use herein. It is anticipated that the covalent bonding of the linker to the polypeptide may be to the uncleaved factor VII which is subsequently cleaved between the 152-153 amino acid positions, or to the cleaved fVIIa.

The term “angiogenesis inhibitor” as used herein refers to a compound or composition that, when administered as an effective dose to an animal or human, will inhibit or reduce the proliferation of vascular endothelial cells, thereby reducing the formation of neovascular capillaries.

Angiogenesis inhibitors may be divided into at least two classes. The first class, direct angiogenesis inhibitors, includes those agents which are relatively specific for endothelial cells and have little effect on tumor cells. Examples of these include soluble vascular endothelial growth factor (VEGF) receptor antagonists and angiostatin.

Indirect inhibitors may not have direct effects on endothelial cells but may down-regulate the production of an angiogenesis stimulator, such as VEGF. (Arbiser et al., Molec. Med. 4:376-383 (1998)). VEGF has been shown to be up-regulated during chemically induced skin carcinogenesis; this is likely due to activation of oncogenes such as H-ras. (Arbiser et al., Proc. Natl. Acad. Sci. U.S.A. 94:861-866 (1997)); (Larcher et al., Cancer Res. 56:5391-5396 (1996)); (Kohl et al., Nature Med. 1:792-797 (1995)). Examples of indirect inhibitors of angiogenesis include inhibitors of ras-mediated signal transduction, such as farnesyltransferase inhibitors.

Direct inhibition of endothelial cell proliferation can be assayed in cell culture systems, in which the effects of specific factors which control the complex process of angiogenesis can be studied. Effects discovered in such in vitro systems can then be studied in in vivo systems as described, for example, by Kenyon et al., Invest. Ophthalmol. 37:1625-1632 (1996).

The term “curcumin (diferuloylmethane)” and certain of its analogs, together termed “curcuminoids,” as used herein, refers to well known natural product, recognized as safe for ingestion by and administration to mammals including humans. (Bille et al., Food Chem. Toxicol. 23:967-971 (1985)). The term “curcuminoid” as used herein also refers to synthetic curcumin derivatives such as, but not limited to those disclosed in PCT Application Serial No. WO 01/40188 incorporated herein by reference in its entirety.

Curcumin is a yellow pigment found in the rhizome of Curcuma longa, the source of the spice turmeric. Turmeric has been a major component of the diet of the Indian subcontinent for several hundred years, and the average daily consumption of curcumin has been found to range up to 0.6 grams for some individuals, without reported adverse effects. Food-grade curcumin consists of the three curcuminoids in the relative amounts: 77% curcumin, 17% demethoxycurcumin, and 3% bisdemethoxycurcumin.

The fully saturated derivative tetrahydrocurcumin is also included in the term curcuminoid. Curcumin can be obtained from many sources, including for example Sigma-Aldrich, Inc. The curcumin analogs demethoxycurcumin, bisdemethoxycurcumin and tetrahydrocurcumin can also be obtained from many sources, or readily prepared from curcumin by those skilled in the art.

Curcumin has been used in indigenous Indian medicine for several hundred years, as a topical agent for sprains and inflammatory conditions, in addition to oral use to promote health and treat digestive and other disorders. Absorption of ingested or orally administered curcumin is known to be limited, and absorbed curcumin is rapidly metabolized. (Govindarajan, CRC Critical Rev. Food Sci Nutr. 12:199-301 (1980); Rao et al., Indian J. Med. Res. 75:574-578 (1982)).

Numerous effects of the ingestion or oral administration of the curcuminoids have been reported, based on controlled research, population studies, case reports and anecdotal information. Evidence of chemopreventive activity of curcumin administered orally has led to clinical trials sponsored by the National Cancer Institute, regarding prevention of cancer. (Kelloff et al., J. Cell. Biochem. Suppl. 26:1-28 (1996)). Oral administration of curcumin to mice treated with skin and colon chemical carcinogens has been shown to result in a decreased incidence and size of induced tumors compared with control mice. (Huang, et al., Cancer Res. 54:5841-5847 (1994); Huang et al., Carcinogenesis 16:2493-2497 (1995); Huang et al., Cancer Lett. 64:117-121; Rao et al., Cancer Res. 55:259-266 (1995); Conney et al., Adv Enzyme Regul. 31:385-396 (1991)).

Huang, et al. found that the oral administration of three curcuminoid compounds curcumin, demethoxycurcumin and bisdemethoxycurcumin were able to inhibit phorbol ester-stimulated induction of ornithine decarboxylase and promotion of mouse skin initiated with 7,12-dimethylbenzanthracene (DMBA). These compounds also inhibited phorbol ester-mediated transformation of JB6 cells. The saturated derivative tetrahydrocurcumin was less active than the unsaturated analogs in these assays. Huang et al., Carcinogenesis 16:2493-2497 (1995).

The mechanism or mechanisms of curcumin's chemopreventive activities were not previously understood, although it was recognized as an antioxidant and was known to exhibit antimutagenic activity in the Ames Salmonella test and to produce biochemical effects similar to those of the polyphenols, chemopreventive agents found in green tea. Stoner, J. Cell. Biochem. Suppl. 22:169-180 (1995). Curcumin has been demonstrated to inhibit several signal transduction pathways, including those involving protein kinase, the transcription factor NF-κB, phospholipase A2 bioactivity, arachidonic acid metabolism, antioxidant activity, and epidermal growth factor (EGF) receptor autophosphorylation. Lu et al., Carcinogenesis 15:2363-2370 (1994); Singh et al., J. Biol. Chem. 270:24995-25000 (1995); Huang et al., Proc. Natl. Acad. Sci. U.S.A. 88:5292-5296 (1991); Korutla et al., Carcinogenesis 16:1741-1745 (1995); Rao et al., Carcinogenesis 14:2219-2225 (1993).

Because of the complexity of the factors that regulate or effect angiogenesis, and their specific variation between tissues and according to circumstances, the response to a specific agent may be different or opposite, in different tissues, under different physiological or pathological conditions and between in vitro and in vivo conditions. For example, U.S. Pat. No. 5,401,504 to Das et al., discloses that oral or topical administration of turmeric to animals including humans promotes wound healing, and postulates that it acts in part through stimulation of angiogenesis, although this postulate was not experimentally verified. Administration of curcumin has been reported to inhibit smooth muscle cell proliferation in vitro. Huang et al., European J. Pharmac. 221:381-384 (1992). U.S. Pat. No. 5,891,924 to Aggarwal discloses that oral administration of curcumin to animals inhibits activation of the transcription factor NF-κB, and claims its use in pathophysiological states, particularly specific conditions involving the immune system. Several biochemical actions of curcumin were studied in detail, but no single action was reported to be responsible for these effects of curcumin. Singh et al. in Cancer Lett. 107:109-115 (1996) reported that curcumin inhibits in vitro proliferation of human umbilical vein endothelial cells (HUVEC) and suggested that it might have anti-angiogenic activity. However, this inhibition was independent of basic fibroblast growth factor stimulation of the proliferation of endothelial cells, and in vivo studies were not reported. Inhibition by curcumin of HUVEC growth and formation of tube structures on Matrigel, in a model of capillary formation, has been ascribed to modulation of metalloproteinases of the HUVEC. (Thaloor et al., Cell Growth Differ. 9:305-312 (1998)).

The term “prodrug” is intended to encompass compounds which, under physiological conditions, may be converted into a pharmaceutically active curcuminoid of the present invention. A common method for making a prodrug is to select moieties which are hydrolyzed under physiological conditions to provide the desired biologically active drug. In other embodiments, the prodrug may be converted by an enzymatic activity of the recipient animal or cell.

The terms “methylketone” and “chloromethylketone” as used herein refer to the carboxy terminus reactive moiety that may form the covalent bond between a peptide linker and an amino acid side chain of a recipient polypeptide. During the linkage reaction, the chloro group is removed. Thus, the unlinked peptidyl linker will have a chloromethylketone moiety and the covalently attached peptide will have a methylketone moiety without a halogen atom thereon.

The term “aliphatic group” as used herein refers to a straight-chain, branched-chain, or cyclic aliphatic hydrocarbon group and includes saturated and unsaturated aliphatic groups, such as an alkyl group, an alkenyl group, and an alkynyl group.

The term “alkyl” as used herein refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. In preferred embodiments, a straight chain or branched chain alkyl has 30 or fewer carbon atoms in its backbone (e.g., C₁-C₃₀ for straight chain, C₃-C₃₀ for branched chain), and more preferably 20 or fewer. Likewise, preferred cycloalkyls have from 3-10 carbon atoms in their ring structure, and more preferably have 5, 6 or 7 carbons in the ring structure. Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, alkylaminos, carbonyl-substituted alkyls, —CF₃, —CN, and the like.

The term “aralkyl”, as used herein, refers to an alkyl group substituted with an aryl group (e.g., an aromatic or heteroaromatic group).

The terms “alkenyl” and “alkynyl” refer to unsaturated aliphatic groups analogous in length and possible substitution to the alkyls described above, but that contain at least one double or triple bond respectively.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. Throughout the application, preferred alkyl groups are lower alkyls. In preferred embodiments, a substituent designated herein as alkyl is a lower alkyl.

The term “aryl” as used herein includes 5-, 6- and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The term “aryl” refers to both substituted and unsubstituted aromatic rings. The aromatic ring can be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, cycloalkynyls, aryls and/or heterocyclyls.

The terms ortho, meta and para apply to 1,2-, 1,3- and 1,4-disubstituted benzenes, respectively. For example, the names 1,2-dimethylbenzene and ortho-dimethylbenzene are synonymous.

The terms “heterocyclyl” or “heterocycle” refer to 4- to 10-membered ring structures, more preferably 3- to 7-membered rings, whose ring structures include one to four heteroatoms. Heterocycles can also be polycycles. Heterocyclyl groups include, for example, thiophene, thianthrene, furan, pyran, isobenzofuran, chromene, xanthene, phenoxathin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, quinoline, pteridine, carbazole, carboline, phenanthridine, acridine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The terms “polycyclyl” or “polycyclic group” refer to two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “carbocycle”, as used herein, refers to an aromatic or non-aromatic ring in which each atom of the ring is carbon.

The phrase “fused ring” is art recognized and refers to a cyclic moiety which can comprise from 4 to 8 atoms in its ring structure, and can also be substituted or unsubstituted, (e.g., cycloalkyl, a cycloalkenyl, an aryl, or a heterocyclic ring) that shares a pair of carbon atoms with another ring. To illustrate, the fused ring system can be a isobenzofuran and a isobenzofuranone.

As used herein, the term “nitro” means —NO₂; the term “halogen” designates —F, —Cl, —Br or —I; the term “sulfhydryl” means —SH; the term “hydroxyl” means —OH; and the term “sulfonyl” means —SO₂—.

The terms “amine” and “amino” are art-recognized and refer to both unsubstituted and substituted amines. The term “alkylamine” as used herein means an amine group, as defined above, having a substituted or unsubstituted alkyl attached thereto.

The term “amido” is art recognized as an amino-substituted carbonyl.

The term “alkylthio” refers to an alkyl group, as defined above, having a sulfur radical attached thereto. In preferred embodiments, the “alkylthio” moiety is represented by one of —S-alkyl, —S-alkenyl, —S-alkynyl, and —S—(CH₂)ₘ. Representative alkylthio groups include methylthio, ethylthio, and the like.

The terms “alkoxyl” or “alkoxy” as used herein refers to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, —O-alkynyl, —O—(CH₂)ₘ.

Analogous substitutions can be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

As used herein, the definition of each expression, e.g. alkyl, m, n, etc., when it occurs more than once in any structure, is intended to be independent of its definition elsewhere in the same structure.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group or other stereogenic centers. All such isomers, as well as mixtures thereof, are intended to be included in this invention. Likewise certain compounds can display overall molecular asymmetry without stereogenic centers leading to sterioisomers

If, for instance, a particular enantiomer of a compound of the present invention is desired, it may be prepared by asymmetric synthesis, or by derivitization with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts can be formed with an appropriate optically-active acid or base, followed by resolution of the diastereomers thus formed by fractional crystallization or chromatographic means well known in the art.

The term “antibody” as used herein refers to polyclonal and monoclonal antibodies and fragments thereof, and immunologic binding equivalents thereof that are capable of selectively binding to a region of tissue factor. The term “antibody” refers to a homogeneous molecular entity, or a mixture such as a polyclonal serum product made up of a plurality of different molecular entities, and may further comprise any modified or derivatised variant thereof that retains the ability to specifically bind an epitope. A monoclonal antibody is capable of selectively binding to a target antigen or epitope.

The phrase “therapeutically-effective amount” as used herein means that amount of a compound, material, or composition comprising a curcuminoid linked to a polypeptide such as, but not limited to, fVIIa by means of a tether and a linker according to the present invention, and which is effective for producing some desired therapeutic effect against cancer or other pathological comprising neovascularization.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or an encapsulating material such as liposomes, polyethylene glycol (PEG), PEGylated liposomes, nonoparticles and the like, involved in carrying or transporting the subject curcuminoid-FFRck-fVIIa agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

Abbreviations: Tissue factor, TF; vascular endothelial cell, VEC; vascular endothelial cell growth factor, VEGF; phenylalanine-phenylalanine-arginyl-(chloro) methylketone, FFR-ck; factor VII(a), fVII(a); active site-inactivated fVIIa, fVIIa-i; tissue factor pathway inhibitor, TFPI.

Curcuminoid-Factor VIIa Conjugates

One aspect of the present invention is compositions that comprise a cytotoxic compound covalently bonded to a protein capable of selectively binding to a cell surface maker, and at least one linker for bonding the compound to the protein The compositions of the present invention may also comprise a tether molecule that alone or in conjunction with the linker may serve to bond the cytotoxic compound to the protein.

The present invention, therefore, provides a composition that comprises a curcuminoid covalently linked by means of a tether and a linker to a polypeptide. The polypeptide is capable of selectively binding to a region (preferably an extracellular region) of a cell surface marker that is an integral component of a cell surface membrane of a target cell such as a vascular endothelial cell. In the various compositions of the present invention, the curcuminoid may be covalently bonded to a tether, which preferably is selected from, but not limited to, a dicarboxylic acid or caproyl moiety. An exemplary tether is succinate that may be bonded to a curcuminoid by the addition of succinic anhydride, as described in Example 2, below. It is, however, also considered to be within the scope of the present invention for any suitable therapeutic compound including, but not limited to curcumin analogs, anticancer drugs or cardiovascular agents, to be conjugated to a linker and a protein by the methods of the present invention, thereby reducing a required effective dose of the agent or drug and to reduce undesirable side effects, by directing the conjugated therapeutic agent to a selected target cell having a surface-exposed marker such as factor.

A suitable polypeptide for use in the compositions of the present invention may be any polypeptide that can selectively bind to a cell surface marker such as, for example, an extracellular region of surface bound tissue factor and which, when so bound, may then be internalized by the targeted cell. Suitable polypeptides include, but are not limited to factor VII or factor VIIa (fVIIa), tissue factor pathway inhibitor (TFPI) or an antibody capable of specifically binding to tissue factor and the like. It is contemplated to be within the scope of the present invention for a suitable polypeptide to be a component polypeptide of fVIIa derived from the amino acid sequence SEQ ID NO: 1 shown in FIG. 1, wherein before conjugation to the linker, the polypeptide may be the uncleaved SEQ ID NO: 1, or cleaved between amino acid positions 152-153 such that the component polypeptide receiving the linker may comprise the amino acid sequence between positions 1 and 152, 153-406 or derivatives thereof, of SEQ ID NO: 1. If the linker is conjugated to the uncleaved amino acid sequence, it is contemplated that the polypeptide may then be cleaved to the fVIIa dipeptide.

The preferred polypeptide for use in the present invention is fVIIa having at least 80% similarity to the amino acid sequence SEQ ID NO:1, as shown in FIG. 1, cleaved between amino acid positions 152 and 153 or truncated derivatives or variants thereof. It is contemplated to be within the scope of the present invention for the fVIIa to be derived from any species, including human fVII. The fVIIa polypeptide for use in the present invention may be truncated to include sequence variations by methods well known to those skilled in the art, including modification of cloned nucleic acid encoding all or part of SEQ ID NO:1, or by proteolytic cleavage of the fVIIa polypeptide, and the like. Any truncation or amino acid substitution will retain the ability of the modified fVIIa and or modified TFPI to selectively bind to tissue factor, be internalized by a target cell and capable of forming a covalent bond with a linker molecule having a chloromethylketone group thereon.

The compositions of the present invention further comprise a linker. One linker suitable for use in the present invention is a peptidyl methylketone linker covalently bonded to the polypeptide, most preferably to the side chain of an amino acid within the catalytic triad of the serine protease domain of fVIIa. In the human and bovine factor VII proteins, the amino acids which form a catalytic “triad” are Ser344, Asp242, and His193, numbering indicating position within the sequence SEQ ID NO:1. The catalytic sites in factor VII from other mammalian species may be determined using presently available techniques including, among others, protein isolation and amino acid sequence analysis. Catalytic sites may also be determined by aligning a sequence with the sequence of other serine proteases, particularly chymotrypsin, whose active site has been previously determined by Sigler et al., J. Mol. Biol., 35:143-164 (1968), incorporated herein by reference, and therefrom determining from said alignment the analogous active site residues. Attachment of the peptidyl linker to this domain will inactivate the serine protease activity, thereby reducing the potential of the composition, when administered to an animal, to induce blood coagulation. In one preferred embodiment of the present invention, at least one linker is covalently bonded to the His 198 position of SEQ ID NO:1.

Peptidyl linkers suitable for use in the present invention, before being bonded to the polypeptide, have a carboxy-terminus chloromethylketone group that may react with a suitable amino acid side chain of the polypeptide, as described in Example 3, below. Preferably, but not necessarily, the carboxy terminal amino acid having the chloromethylketone group thereon is an arginine. Although any peptidyl chain sequence or length may be used in the compositions of the present invention, a suitable peptide is a tripeptide. Preferred peptidyl linkers include, but are not limited to, tyrosine-glycine-arginine-chloromethylketone (YGR-ck); phenylalanine-phenylalanine-arginine-chloromethylketone (FFR-ck), glutamine-glycine-arginine-chloromethylketone (QGR-ck), glutamate-glcine-arginine chloromethylketone (EGR-ck) and the like. A most preferred linker is FFR-ck. The stoichiometry of attachment of the curcuminoid EF24-tether-FFRck to fVIIa is given in Example 4, below.

It will be understood by those of skill in the art that upon covalently attaching the chloromethylketone to the recipient polypeptide, the chloro—moiety is displaced. Accordingly, the term “FFR-ck-VIIa”, for example, refers to FFR-methylketone tripeptidyl linker bonded to fVIIa and not having a chloro—atom attached thereto.

While not wishing to be bound by any one theory, a complex, formed from phenylalanyl-phenylalanyl-arginyl-ck-VIIa (FFR-ck-VIIa) and tissue factor (TF) expressed on the plasma membrane of cancer cells, may be internalized in a FFR-ck-VIIa concentration-dependent manner by ligand-receptor mediated endocytosis. The ligand-receptor complex is endocytosed into early and late endosomes and is delivered to lysosomal vesicles and degraded by lysosomal enzymes.

The peptide selected for use as a linker peptide in the compositions of the present application is also suitable for cleavage by an intracellular hydrolytic activity of the target cell enzyme. When so cleaved, after endocytotic internalization, the curcuminoid attached to the linker may be released from a polypeptide such as fVIIa. The released curcuminoid may then modulate a physiological function of the target cell.

More than ninety novel curcumin analogs (patent pending for all compounds) have been synthesized, as described in PCT application serial number WO 01,40188 incorporated herein by reference in its entirety. Several of these compounds suppress cancer cell VEGF production, but are not cytotoxic to either cancer cells or endothelial cells at concentrations where curcumin is otherwise cytotoxic.

A particularly suitable curcuminoid for use in the compositions of the present invention is 3,5-Bis-(2-fluorobenzylidene)-piperidin-4-one (EF24 having the formula: 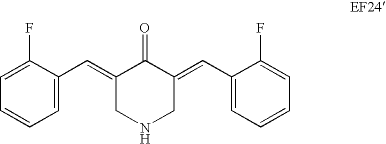
or a salt thereof.

It is contemplated that any curcuminoid such as, but not limited to, those curcuminoids disclosed in PCT application Serial No. 01/40188 incorporated herein by reference in its entirety, may be used in the compositions of the present invention if capable of bonding to a carboxylic or polycaproyl tether by reactions such as described, for example, in Example 2, below. Methods for synthesizing the curcuminoids are also fully disclosed in PCT application Serial No. 01/40188.

One embodiment of the compositions of the present invention, therefore, comprises a protein, wherein the protein selectively binds a surface marker of a target cell, at least one linker covalently bonded to the protein, and a cytotoxic compound bonded to the linker by a hydrolysable bond.

In one embodiment of the compositions of the present invention, the protein selectively binds to tissue factor on the surface of the target cell.

In another embodiment of the compositions of the present invention, the protein is a component polypeptide of a factor VIIa.

In yet another embodiment of the compositions of the present invention, the protein is a component polypeptide of a factor VIIa, and the polypeptide comprises the amino acid sequence between amino acid positions 153 and 406 of SEQ ID NO: 1 or a truncated or modified variant thereof.

In embodiments of the compositions of the present invention, the protein is selected from an antibody and tissue factor pathway inhibitor.

In various embodiments of the compositions of the present invention, the protein is capable of being internalized by the target cell.

In other embodiments of the compositions of the present invention, at least one linker is a peptidyl linker.

In various embodiments of the compositions of the present invention, at least one peptidyl linker is a peptidyl methylketone linker.

In the embodiments of the compositions of the present invention, the composition may further comprise a tether.

In one embodiment of the compositions of the present invention, the linker is a tether.

In the various embodiments of the compositions of the present invention, the hydrolysable bond is selected from the group consisting of a carbamate, an amide, an ester, a carbonate and a sulfonate.

In the embodiments of the compositions of the present invention, at least linker is an arginyl methylketone selected from the group consisting of phenylalanine-phenylalanine-arginine methylketone, tyrosine-glycine-arginine methylketone, glutamine-glycine-arginine methylketone, glutamate-glycine-arginine methylketone and phenylalanine-proline-arginine methylketone.

In other embodiments of the compositions of the present invention, a linker is selected from tyrosine-glycine-arginine methylketone and phenylalanine-phenylalanine-arginine methylketone.

In one embodiment of the compositions of the present invention, the linker is phenylalanine-phenylalanine-arginine methylketone.

In another embodiment of the compositions of the present invention, the linker is tyrosine-glycine-arginine methylketone.

In still other embodiments of the compositions of the present invention, a linker is covalently bonded to an amino acid side chain within a serine protease active site of factor VIIa, thereby inactivating the serine protease active site.

In various embodiments of the compositions of the present invention, the cytotoxic compound may be a curcuminoid having the formula: 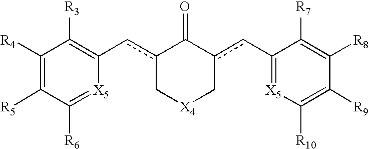
wherein X₄ is (CH₂)ₘ, O, S, SO, SO₂, or NR₁₂, where R₁₂ is H, alkyl, substituted alkyl, acyl, alkoxycarbonyl, aminocarbonyl, alkylaminocarbonyl or dialkylaminocarbonyl; m is 1-7; each X₅ is independently N or C—R₁₁; and each R₃-R₁₁ are independently H, halogen, hydroxyl, alkoxy, CF₃, alkyl, substituted alkyl, alkenyl, alkynyl, cycloalkyl, substituted cycloalkyl, aryl, substituted aryl, alkaryl, arylalkyl, heteroaryl, substituted heteroaryl, heterocycle, substituted heterocycle, amino, alkylamino, dialkylamino, carboxylic acid, carboxylic ester, carboxamide, nitro, cyano, azide. alkylcarbonyl, acyl, or trialkylammonium; and the dashed lines indicate optional double bonds; with the proviso that when X₄ is (CH₂)ₘ, m is 2-6, and each X₅ is C—R₁₁, R₃-R₁₁ are not alkoxy, and when X₄ is NR₁₂ and each X₅ is N, R₃-R₁₀ are not alkoxy, alkyl, substituted alkyl, alkenyl, alkynyl, cycloalkyl, substituted cycloalkyl, aryl, substituted aryl, alkaryl, arylalkyl, heteroaryl, substituted heteroaryl, amino, alkylamino, dialkylamino, carboxylic acid, or alkylcarbonyl, and wherein the stereoisomeric configurations include enantiomers and diastereoisomers, and geometric (cis-trans) isomers.

In some embodiments of the compositions of the present invention, X₄ is selected from the group consisting of —NH and —NR₁₂, and R₃-R₁₀ may be selected from hydroxyl and —NHR₁₂.

In one embodiment of the compositions of the present invention, the cytotoxic compound is a curcuminoid having the formula:

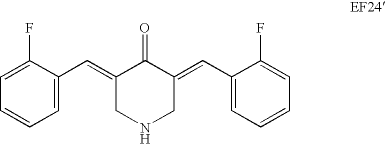

In yet other embodiments of the compositions of the present invention, the tether is selected from the group consisting of a dicarboxylic acid, a disulfonic acid, an omega-amino carboxylic acid, an omega-amino sulfonic acid, an omega-amino carboxysulfonic acid, or a derivative thereof, wherein the tether comprises 2-6 carbons, and wherein the tether is capable of forming a hydrolysable bond.

In one embodiment of the compositions of the present invention, the tether comprises a dicarboxylic acid and, in another embodiment, the tether is succinate.

The present invention further provide methods of producing a curcuminoid-polypeptide conjugate, comprising the steps of (a) synthesizing a product comprising a curcuminoid having a tether covalently bonded thereto, wherein the curcuminoid has the formula: 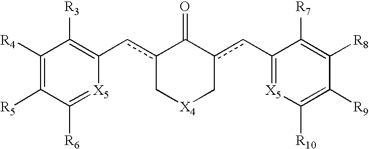
wherein X₄ is (CH₂)ₘ, O, S, SO, SO₂, or NR₁₂, where R₁₂ is H, alkyl, substituted alkyl, acyl, alkoxycarbonyl, aminocarbonyl, alkylaminocarbonyl or dialkylaminocarbonyl, m is 1-7, each X₅ is independently N or C—R₁₁, and each R₃-R₁₁ are independently H, halogen, hydroxyl, alkoxy, CF₃, alkyl, substituted alkyl, alkenyl, alkynyl, cycloalkyl, substituted cycloalkyl, aryl, substituted aryl, alkaryl, arylalkyl, heteroaryl, substituted heteroaryl, heterocycle, substituted heterocycle, amino, alkylamino, dialkylamino, carboxylic acid, carboxylic ester, carboxamide, nitro, cyano, azide. alkylcarbonyl, acyl, or trialkylammonium; and the dashed lines indicate optional double bonds; with the proviso that when X₄ is (CH₂)ₘ, m is 2-6, and each X₅ is C—R₁₁, R₃-R₁₁ are not alkoxy, and when X₄ is NR₁₂ and each X₅ is N, R₃-R₁₀ are not alkoxy, alkyl, substituted alkyl, alkenyl, alkynyl, cycloalkyl, substituted cycloalkyl, aryl, substituted aryl, alkaryl, arylalkyl, heteroaryl, substituted heteroaryl, amino, alkylamino, dialkylamino, carboxylic acid, or alkylcarbonyl, and wherein the stereoisomeric configurations include enantiomers and diastereoisomers, and geometric (cis-trans) isomers, (b) providing a peptidyl chloromethylketone linker, (c) bonding covalently the product of step (a) and the linker, and (d) covalently bonding the composition of step (c) to a polypeptide capable of selectively binding to tissue factor on the surface of a target cell.

In one embodiment of the methods of the present invention, the method comprises the steps of synthesizing a product comprising a cytotoxic compound, bonding covalently the product of step (a) and the linker, and covalently bonding at least one molecule of the composition of step (b) to a protein capable of selectively binding to a surface marker of a target cell.

In one embodiment of this aspect of the present invention, step (a) comprises reacting the curcuminoid with a tether selected from the group consisting of a dicarboxylic acid, a disulfonic acid, an omega-amino carboxylic acid, an omega-amino sulfonic acid, an omega-amino carboxysulfonic acid, or a derivative thereof, wherein the tether comprises 2-6 carbons, and wherein the tether is capable of forming a hydrolysable bond.

In various embodiments of the method of the present invention, X₄ is selected from the group consisting of —NH and —NR₁₂, and R₃-R₁₀ can be selected from hydroxyl and —NHR₁₂.

In one embodiment of the method of the present invention, the cytotoxic compound has the formula:

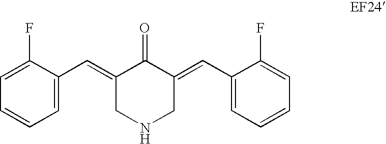

In another embodiment of the method of the present invention, step (a) comprises reacting the cytotoxic compound with a dicarboxylic anhydride. In yet another embodiment of the compositions of the present invention, the dicarboxylic anhydride is succinic anhydride, and

In one embodiment of the method of the present invention, the product of step (a) has the formula: 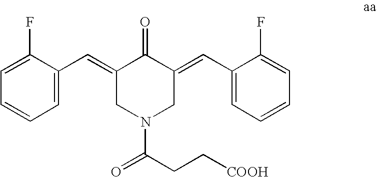
and in yet another embodiment, step (b) comprises providing a peptidyl linker. In various embodiments, step (b) comprises the steps of reacting a composition having the formula: 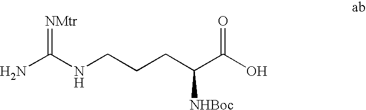
with isopropyl chloroformate and ethereal diazomethane, thereby producing a compound having the formula: 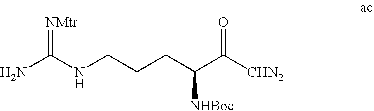
reacting a compound having the formula: 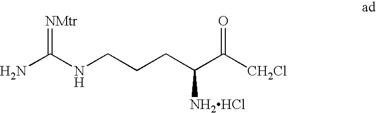
with N-Boc-Phe-Phe-OH, isopropyl chloroformate, and a base; thereby producing a compound having the formula: 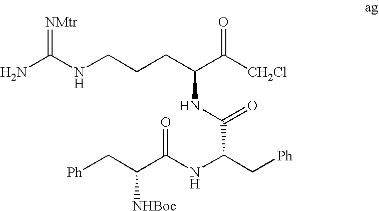
deprotecting compound ag, thereby producing a compound having the formula:

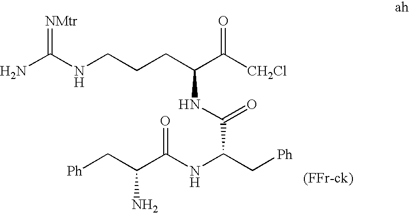

In one embodiment of the method of the present invention, the composition of step (b) has the formula:

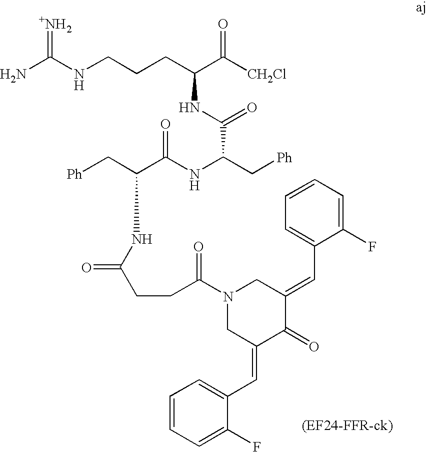

In one embodiment of the method of the present invention, the protein is a component polypeptide of a factor VIIa.

In another embodiment of the method of the present invention, at least molecule of the composition of step (b) is covalently bonded to an amino acid of the serine protease active site of factor VIIa, thereby inactivating the active site.

In yet another embodiment of the method of the present invention, the amino acid is the His193 of SEQ ID NO: 1.

Pharmaceutical Compositions

Another aspect of the present invention provides pharmaceutically acceptable compositions that comprise a therapeutically-effective amount of a curcuminoid linked to a tissue a factor-specific polypeptide such as described above, formulated together with one or more pharmaceutically acceptable carriers (additives) and/or diluents for use as a therapeutic agent for the treatment of a pathological condition of an animal or human such as a cancer or a neovascular based disease.

The efficacy of the curcuminoids suitable for use in the present invention as cytotoxic agents effective against cancer cells is fully described in PCT Application Serial No. WO 01/40188 incorporated herein in its entirety.

The cytotoxic effects of the novel curcumin-FFRck-fVIIa constructs of the present invention were tested on human prostate cancer cells (Example 5, below), breast cancer (Example 6) and melanoma cells (Example 7), umbilical cord vascular endothelial cells (HUVECs) (Example 8) and murine vascular endothelial cells immortalized by transfection of SV40 large T antigen (MS-1 Cells). MS-1 cells are regarded as benign because the cells, when in nude mice, remain as small tumors a few millimeters in diameter during the entire life span of the mice, and do not metastasize. Normal HUVEC cells induced to express high-levels of tissue factor by exposure to phorbol ester are susceptible to the cytotoxic effect of the EF24-FFR-ck-fVIIa conjugate, as shown in Example 9, below.

As described in detail below, the pharmaceutical compositions of the present invention may be specially formulated for administration in solid or liquid form, including those adapted for oral administration or parenteral administration, for example, by subcutaneous, intramuscular or intravenous injection as, for example, a sterile solution or suspension.

One aspect of the present invention, therefore is a pharmaceutical composition comprising a protein, wherein the protein selectively binds a surface marker of a target cell, and wherein the protein is covalently bonded to at least one linker, wherein each linker has a cytotoxic compound bonded thereto, and wherein said cytotoxic compound is covalently linked by hydrolysable bond to the linker, and a pharmaceutically acceptable carrier.

In various embodiments of this aspect of the present invention, the pharmaceutical composition further comprises a tether covalently linked by hydrolysable bond to the cytotoxic compound.

Also in various embodiments of this aspect, the hydrolysable bond is selected from the group consisting of a carbamate, an amide, an ester, a carbonate and a sulfonate.

In yet other embodiments of the pharmaceutical composition of the present invention, the tether is selected from the group consisting of a dicarboxylic acid, a disulfonic acid, an omega-amino carboxylic acid, an omega-amino sulfonic acid, an omega-amino carboxysulfonic acid, or a derivative thereof, wherein the tether comprises 2-6 carbons, and wherein the tether is capable of forming a hydrolysable bond.

In various embodiments of this aspect of the present invention, at least one linker is an arginyl methylketone selected from the group consisting of phenylalanine-phenylalanine-arginine methylketone, tyrosine-glycine-arginine methylketone, glutamine-glycine-arginine methylketone, glutamate-glycine-arginine methylketone and phenylalanine-proline-arginine methylketone.

Also in various aspects of the present invention, the cytotoxic compound is a curcuminoid having the formula:

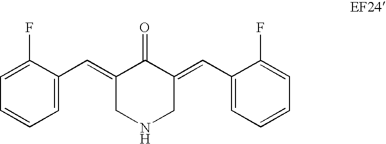

In one embodiment of the pharmaceutical composition of the present invention, the pharmaceutically composition is formulated in a pharmaceutically effective dosage amount.

In one embodiment of the pharmaceutical composition of the present invention, the protein is a component polypeptide of a factor VIIa.

In yet another embodiment of the pharmaceutical composition of the present invention, the pharmaceutical composition is formulated for intravenous infusion.

Pharmaceutical Administration

The regimen for any patient to be treated with a pharmaceutical composition mentioned herein should be determined by those skilled in the art. The daily dose to be administered in therapy can be determined by a physician and will depend on the particular compound employed, on the route of administration and on the weight and the condition of the patient.

The pharmaceutical composition of the present invention can be administered in a single dose, but it can also be given in multiple doses with intervals between successive doses depending on the dose given and the condition of the patient.

The pharmaceutical composition of the present invention may be administered intravenously or it may be administered by continuous or pulsatile infusion, preferably administered by intraveneous injections.

For the treatment of skin disorders, the angiogenesis inhibitors of the present invention are preferably administered systemically. For treatment of certain disorders, however, the curcuminoid-tether-linker-fVIIa may be applied topically in diseases or pathologic conditions of the skin, or locally in other tissues, to treat cancer, pre-malignant conditions and other diseases and conditions in which angiogenesis occurs.

The administration of these agents topically or locally may also used to prevent initiation or progression of such diseases and conditions. For example, a curcuminoid formulation may be administered topically or by instillation into a bladder if a biopsy indicated a pre-cancerous condition or into the cervix if a Pap smear was abnormal or suspicious.

The angiogenesis inhibiting formulation is administered as required to alleviate the symptoms of the disorder. Assays can be performed to determine an effective amount of the agent, either in vitro and in vivo. Representative assays are described in the examples provided below. Other methods are known to those skilled in the art, and can be used to determine an effective dose of these and other agents for the treatment and prevention of diseases or other disorders as described herein.

Conventional techniques for preparing pharmaceutical compositions which can be used according to the present invention are, for example, described in Remington's Pharmaceutical Sciences, 1985.

In short, pharmaceutical preparations suitable for use according to the present invention are made by mixing the pharmaceutical composition, preferably in purified form, with suitable adjuvants and a suitable carrier or diluent. Suitable physiological acceptable carriers or diluents include sterile water and saline. Suitable adjuvants, in this regard, include calcium, proteins (e.g. albumins), or other inert peptides (e.g. glycylglycine) or amino acids (e.g. glycine, or histidine) to stabilise the purified factor VIIa. Other physiological acceptable adjuvants are non-reducing sugars, cyclodextrins (cyclic carbohydrates derived from starch), polyalcohols (e.g. sorbitol, mannitol or glycerol), polysaccharides such as low molecular weight dextrins, detergents (e.g. polysorbate) and antioxidants (e.g. bisulfite and ascorbate). The adjuvants are generally present in a concentration of, but not limited to, from 0.001 to 4% w/v. The pharmaceutical preparation may also contain protease inhibitors, e.g. aprotinin, and preserving agents.

The preparations may be sterilized by, for example, filtration through a bacteria-retaining filter, by incorporating sterilizing agents into the compositions, by irradiating the compositions. They can also be manufactured in the form of sterile solid compositions which can be dissolved in sterile water, or some other sterile medium suitable for injection prior to or immediately before use.

Certain embodiments of the present invention comprise curcuminoids or derivatives thereof that may contain a basic functional group, such as amino or alkylamino, and are, thus, capable of forming pharmaceutically-acceptable salts with pharmaceutically-acceptable acids. The term “pharmaceutically-acceptable salts” in this respect, refers to the relatively non-toxic, inorganic and organic acid addition salts of curcuminoids. These salts can be prepared in situ during the final isolation and purification of the compounds of the invention, or by separately reacting a purified compound of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, for example, Berge et al. (1977) “Pharmaceutical Salts”, J. Pharm. Sci. 66:1-19).

In other cases, the compounds of the present invention may contain one or more acidic functional groups and, thus, are capable of forming pharmaceutically-acceptable salts with pharmaceutically-acceptable bases. The salts can likewise be prepared in situ during the final isolation and purification of the curcuminoid containing composition of the present invention, or by separately reacting derivatives comprising carboxylic or sulfonic groups with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

It is contemplated that formulations of the present invention may include those suitable for parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the curcuminoid derivatives thereof which produces a therapeutic effect. Generally, out of one hundred percent, this amount will range from about 0.1 percent to about 99.5 percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a curcuminoid-linker-fVIIa conjugate of the present invention with liquid carriers, or finely divided solid carriers, or both, and then, if necessary, shaping the product.

Pharmaceutical compositions of this invention suitable for parenteral administration may comprise one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and other antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as polyethylene glycol (PEG), aluminum monostearate and gelatin.

In some cases, in order to prolong the effect of a drug, it is desirable to slow the absorption of the drug from subcutaneous or intramuscular injection. This may be accomplished by coupling to PEG, the use of a liquid suspension of crystalline or amorphous material having poor water solubility. The rate of absorption of the drug then depends upon its rate of dissolution which, in turn, may depend upon size, form and amount of PEG, crystal size and crystalline form. Alternatively, delayed absorption of a parenterally-administered drug form is accomplished by dissolving or suspending the drug in an oil vehicle.

Injectable depot forms are made by forming microencapsuled matrices of the subject peptides or peptidomimetics in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

The pharmaceutical compositions are intended for parenteral, topical or local administration for prophylactic and/or therapeutic treatment. Most preferably, the pharmaceutical compositions are administered parenterally, i.e., intravenously, so that the compositions of the present invention may be rapidly transported to a selected target cell such as a cancer cell or neovascular endothelial cell. Thus, this invention provides compositions for parenteral administration which comprise a solution of the modified fVII molecules dissolved in an acceptable carrier, preferably an aqueous carrier. A variety of aqueous carriers may be used, e.g., water, buffered water, 0.4% saline, 0.3% glycine and the like. The modified fVIIa molecules can also be formulated into liposome preparations for delivery or targeting to sites of injury. Liposome preparations are generally described in, e.g., U.S. Pat. No. 4,837,028, U.S. Pat. No. 4,501,728, and U.S. Pat. No. 4,975,282, incorporated herein by reference. The compositions may be sterilized by conventional, well known sterilization techniques. The resulting aqueous solutions may be packaged for use or filtered under aseptic conditions and lyophilized, the lyophilized preparation being combined with a sterile aqueous solution prior to administration. The compositions may contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride, etc. The concentration of modified factor VII in these formulations can vary widely, i.e., from less than about 0.5%, usually at or at least about 1% to as much as 15 or 20% by weight and will be selected primarily by fluid volumes, viscosities, etc., in accordance with the particular mode of administration selected.

Thus, a desirable exemplary pharmaceutical composition for intravenous infusion could be made up to contain 0.05-5 mg/kg body weight (in rats) or 0.05-10 mg/kg human adult in 250 ml of sterile Ringer's solution, and 10 mg of modified factor VII. Actual methods for preparing parenterally administrable compounds will be known or apparent to those skilled in the art and are described in more detail in for example, Remington's Pharmaceutical Science, 16th ed., Mack Publishing Company, Easton, Pa. (1982), which is incorporated herein by reference.

Yet another aspect of the present invention, is methods of modulating a physiological function of a target cell, comprising the steps of contacting a target cell having a surface marker thereon with a composition comprising a cytotoxic compound-protein conjugate, wherein the composition selectively binds to the surface marker and is internalized, thereby releasing the cytotoxic compound from the protein; and modulating the physiological function of the target cell.

In one embodiment of the method of the present invention, the surface marker is tissue factor.

In various embodiments of the method of the present invention, the physiological function is proliferation of the cell, and wherein proliferation is reduced.

In the embodiments of this method of the present invention, the target cell can be selected from a vascular endothelial cell, a vascular smooth muscle cell, a tumor cell, a monocyte, a macrophage and a microparticle. In one embodiment, the target cell is a vascular endothelial cell. In yet another embodiment, the target cell is a vascular smooth muscle cell.

In yet other embodiments of this method of the present invention, the vascular endothelial cell can be selected from the group consisting of an isolated vascular endothelial cell, a capillary endothelial cell, a venal endothelial cell, an arterial endothelial cell and a neovascular endothelial cell of a tumor.

In other embodiments of this aspect of the present invention, the composition further comprises a pharmaceutically acceptable carrier.

In still another embodiment of this method, the target cell is an cultured cell.

Various embodiments of this method of the present invention further comprise the step of delivering the composition to an animal or human having the target cell, wherein the composition is delivered to an animal or human by a route selected from the group consisting of topical intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, intrasternal injection and infusion.

Yet another aspect of the present invention is a method of selectively delivering a cytotoxic compound to a target cell, comprising the steps of contacting a target cell having a surface marker thereon with a composition according to claim 1; and binding the composition to the surface marker on the target cell, whereby the composition is internalized by the target cell, thereby delivering the cytotoxic compound to the interior of the target cell.

In this aspect of the present invention, the therapeutic preparation may further comprise a pharmaceutically acceptable carrier.

In one embodiment of this method of the present invention, the cytotoxic compound is a curcuminoid having the formula: 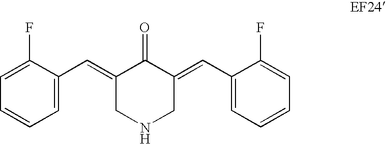
and wherein the protein is a component polypeptide of factor VIIa.

Still yet another aspect of the present invention is a method of modulating a pathological condition in an animal or human, comprising the step of administering to an animal or human subject having a pathological condition an effective dose of a composition comprising a cytotoxic compound-protein conjugate according to the present invention, thereby reducing the proliferation of a target cell capable of expressing surface-bound marker, and thereby modulating the pathological condition of the patient subject.

In one embodiment of this method of the present invention, the surface marker of the target cell is tissue factor.

In various embodiments of this method of the present invention, the pathological condition is selected from the group consisting of cancer, hypercoagulapathy, restenosis, diabetic retinopathy, rheumatoid arthritis and a skin disorder inflammation.

Also in various embodiments of this aspect of the present invention, the pathological condition is a cancer selected from the group consisting of leukemia, breast cancer, lung cancer, liver cancer, melanoma and prostrate cancer.

In one embodiment of this method of the present invention, the target cell is a vascular endothelial cell.

In yet another embodiment of this method of the present invention, the target cell is a vascular smooth muscle cell.

In still another embodiment of this method of the present invention, the target cell is a cancer cell.

In another embodiment of this method of the present invention, the composition is antiangiogenic and wherein reducing proliferation of a target cell reduces angiogenesis and, in another embodiment, reducing angiogenesis causes a reduction in a tumor.

Restenois

Recent advances in the treatment of coronary vascular disease include the use of mechanical interventions to either remove or displace offending plaque material in order to re-establish adequate blood flow through the coronary arteries. Despite the use of multiple forms of mechanical interventions, including balloon angioplasty, reduction atherectomy, placement of vascular stents, laser therapy, or rotoblator, the effectiveness of these techniques remains limited by an approximately 40% restenosis rate within 6 months after treatment.

Restenosis is thought to result from a complex interaction of biological processes including platelet deposition and thrombus formation, release of chemotactic and mitogenic factors, and the migration and proliferation of vascular smooth muscle cells into the intima of the dilated arterial segment. The inhibition of platelet accumulation at sites of mechanical injury can limit the rate of restenosis in human subjects. Inhibition of platelet accumulation at the site of mechanical injury in human coronary arteries is beneficial for the ultimate healing response that occurs. While platelet accumulation occurs at sites of acute vascular injuries, the generation of thrombin at these sites may be responsible for the activation of the platelets and their subsequent accumulation.

The modified fVIIa of the present invention is able to bind to cell-surface tissue factor but has no enzymatic activity. It will, however, act as a competitive antagonist for wild-type fVIIa, thereby inhibiting the subsequent steps in the extrinsic pathway of coagulation leading to the generation of thrombin.

Modified fVIIa molecules of the present invention that maintain tissue factor binding, inhibit platelet accumulation at the site of vascular injury by blocking the production of thrombin and the subsequent deposition of fibrin.

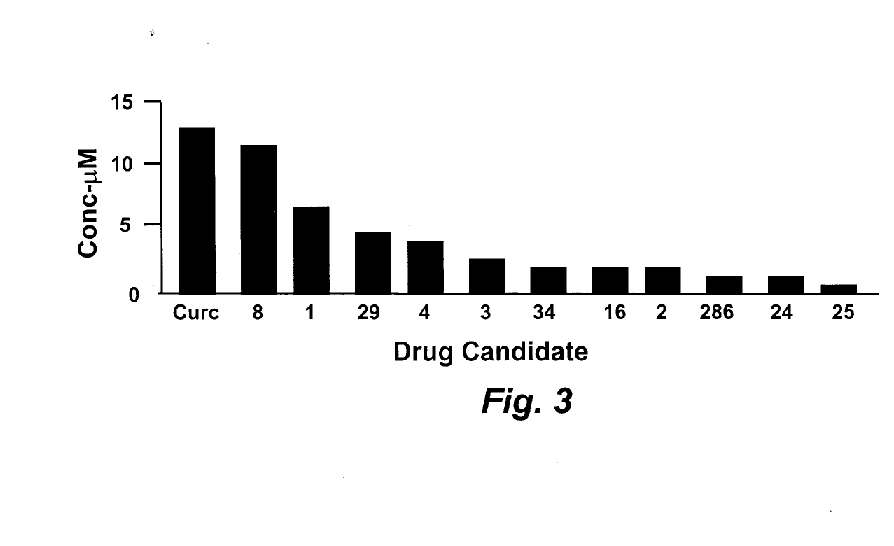

The curcuminoid-linker-fVIIa conjugates of the present invention block thrombin generation and limit platelet deposition at sites of acute vascular injury, and therefore are useful for inhibiting vascular restenosis. The compositions of the present invention may further inhibit restenosis by internalization by proliferating endothelial or smooth muscle cells, thereby delivering curcuminoids such as, but not limited to, EF24, to the cytoplasm of a target cell. The curcuminoids may then directly kill the target cell, as shown in FIG. 3 wherein various candidate curcuminoids including EF24 were added to endothelial cells immortalized with the Ras gene, thereby reducing or eliminating restenosis.

Thus, the compositions and methods of the present invention have a wide variety of uses. For example, they are useful in preventing or inhibiting restenosis following intervention, typically mechanical intervention, to either remove or displace offending plaque material in the treatment of coronary or peripheral vascular disease, such as in conjunction with and/or following balloon angioplasty, reductive atherectomy, placement of vascular stents, laser therapy, rotoblation, and the like. The compounds will typically be administered within about 24 hours prior to performing the intervention, and for as much as 7 days or more thereafter. Administration can be by a variety of routes as further described herein. The preferred route will be direct delivery to a blood vessel, possibly close to the site of restenosis or tissue damage for rapid and specific delivery to tissue factor-bearing cells. The compounds of the present invention can also be administered systemically or locally for the placement of vascular grafts (e.g., by coating synthetic or modified natural arterial vascular grafts), at sites of anastomosis, surgical endarterectomy (typically carotid artery endarterectomy), bypass grafts, and the like. The modified fVIIa also finds use in inhibiting intimal hyperplasia, accelerated atherosclerosis and veno-occlusive disease associated with organ transplantation, e.g., following bone marrow transplantation.

The curcuminoid-linker-fVIIa conjugates of the present invention are particularly useful in the treatment of intimal hyperplasia or restenosis due to acute vascular injury. Acute vascular injuries are those which occur rapidly (i.e. over days to months), in contrast to chronic vascular injuries (e.g. atherosclerosis) which develop over a lifetime. Acute vascular injuries often result from surgical procedures such as vascular reconstruction, wherein the techniques of angioplasty, endarterectomy, atherectomy, vascular graft emplacement or the like are employed. Hyperplasia may also occur as a delayed response in response to, e.g., graft emplacement or organ transplantation. Since conjugated fVIIa is more selective than heparin, generally binding only tissue factor which has been exposed at sites of injury, and because modified fVII does not destroy other coagulation proteins, it will be more effective and less likely to cause bleeding complications than heparin when used prophylactically for the prevention of deep vein thrombosis. The dose of modified fVII for prevention of deep vein thrombosis is in the range of about 50 μg to 500 mg/day, more typically 1 mg to 200 μg/day, and more preferably 10 to about 175 μg/day for a 70 kg patient, and administration begin at least about 6 hours prior to surgery and continue at least until the patient becomes ambulatory. The dose of the curcuminoid-fVIIa conjugates of the present invention in the treatment for restenosis will vary with each patient but will generally be in the range of those suggested above.

Although preferred embodiments of the invention have been described using specific terms, devices, and methods, such description is for illustrative purposes only. The words used are words of description rather than of limitation. It is to be understood that changes and variations may be made by those of ordinary skill in the art without departing from the spirit or the scope of the present invention, which is set forth in the appended claims. In addition, it should be understood that aspects of the various embodiments may be interchanged both in whole or in part.

The present invention is further illustrated by the following examples, which are provided by way of illustration and should not be construed as limiting. The contents of all references, published patents, and patents cited throughout the present application are also hereby incorporated by reference in their entireties.

### EXAMPLE 1

### Factor VII (fVIIa)

Human purified factor VIIa suitable for use in the present invention is preferably made by DNA recombinant technology, e.g. as described by Hagen et al., Proc. Natl. Acad. Sci. USA 83: 2412-2416, (1986) or as described in European Patent No. 200.421. Factor VIIa produced by recombinant technology may be authentic factor VIIa or a more or less modified factor VIIa provided that such factor VIIa has substantially the same biological activity for blood coagulation as authentic factor VIIa. Such modified factor VIIa may be produced by modifying the nucleic acid sequence encoding factor VII either by altering the amino acid codons or by removal of some of the amino acid codons in the nucleic acid encoding the natural fVII by known means, e.g. by site-specific mutagenesis.

Factor VII may also be produced by the methods described by Broze & Majerus, J. Biol. Chem. 255 (4): 1242-1247, (1980) and Hedner & Kisiel, J. Clin. Invest. 71: 1836-1841, (1983). These methods yield factor VII without detectable amounts of other blood coagulation factors. An even further purified factor VII preparation may be obtained by including an additional gel filtration as the final purification step. Factor VII is then converted into activated fVIIa by known means, e.g. by several different plasma proteins, such as factor XIIa, IXa or Xa. Alternatively, as described by Bjoern et al. (Research Disclosure, 269 September 1986, pp. 564-565), factor VII may be activated by passing it through an ion-exchange chromatography column, such as Mono QR™. (Pharmacia Fine Chemicals) or the like.

### EXAMPLE 2

### Synthesis of Curcumin Analogs and Coupling of EF24 and FFRck Using a Succinate Tether

Descriptions and synthetic preparations of a series of monocarbonyl curcumin analogs useful in the present invention has been described in PCT Application Serial No. 01/40188 incorporated herein by reference in its entirety.

Synthesis of the conjugate of fVIIa protein and the drug molecule, EF24-FFRck-fVIIa proceeded in three steps. First, an appropriate derivative of EF24 was developed that permitted attachment to the FFR tripeptide. To synthesize EF24, piperidone.hydrate.HCl (2.2 g, 14 mmol) was suspended in glacial CH₃COOH (60 ml). The suspension was saturated with HCl gas until the solution became clear, and then treated with solid 2-fluorobenzaldehyde (5.0 g, 40 mmol). The reaction mixture was stirred at ambient temperature for 48 hrs. The precipitated solid was collected by filtration, washed with cold absolute ethanol, and dried in vacuo to give a bright-yellow crystalline solid (EF24, 4.27 g, 80% yield).

Of several compounds examined, the succinic acid derivative aa (86% yield) was suitable since it retained 50% of the activity of EF24 in cell cytotoxicity assays. To synthesize the succinyl derivative aa of EF24, to a solution of EF24 (0.16 g, 0.5 mmol) in anhydrous CH₂Cl₂ (6 ml) was added succinic anhydride (0.057 g, 0.5 mmol) and Et₃N (101 mg, 1 mmol). The mixture was stirred at room temperature for 3 hrs, diluted with CH₂Cl₂, washed twice with saturated NaHCO₃ (2×10 ml) and brine, dried over anhydrous Na₂SO₄, and relieved of solvent by evaporation. The resulting solid was purified by flash chromatography using benzene/acetone/acetic acid (27:10:0.5) as the eluant to obtain the yellow solid aa (852 mg, mp 145° C., 86% yield).

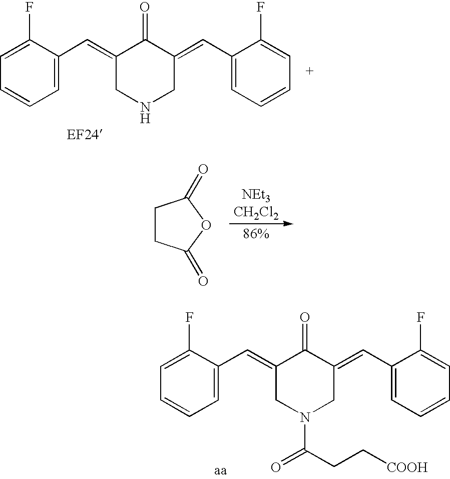

Second, the FFR-ck peptide linker was assembled as shown below. For this step, commercially available Boc-Arg(Mtr)-OH (ab 122 mg, 0.25 mmol) was dissolved in THF (2 ml) and allowed to react with isopropyl chloroformate (1.0 M in toluene, 0.25 ml, 0.25 mmol) in the presence of N-methylmorpholine (25 mg, 0.25 mmol) for 4 hrs at −20° C. The mixture was filtered, and the filtrate was added to 4 ml of ethereal diazomethane. After stirring the reaction solution for 1 hr at 0° C., the solvent was evaporated to obtain the crude product as white needles. These were purified by chromatography using ethyl acetate as the eluant to obtain a white solid, ac (75 mg, 59% yield).

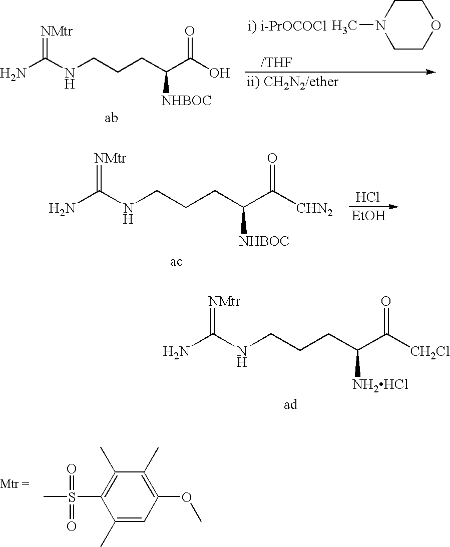

N-Boc-Phe-Phe-OH af (197 mg, 0.4 mmol) was allowed to react with N-methylmorpholine (40 mg, 0.4 mmol) and isopropyl chloroformate (1.0 M in toluene, 0.4 ml, 0.4 mmol) for 10 mins at −20° C. Cold THF (5.72 ml) containing N-methylmorpholine (40 mg, 0.4 mmol) was added to the mixture which was immediately added to Arg(Mtr)CH₂Cl.HCl ad (200 mg, 0.4 mmol) dissolved in DMF (0.92 ml). After stirring for 1 hr at −20° C. and 2 hrs at room temperature, THF (5.6 ml) was added and the mixture was filtered. The filtrate was evaporated and the solid residue purified by column using EtOAc/hexanes (4:1) as the eluant. A white solid was obtained, ag (245 mg, 75% yield). Compound ag (0.05 mmol, 42.5 mg) was dissolved in EtOAc (0.16 ml) and allowed to react with methanolic HCl (0.85 mmol) at room temperature for 3.5 hrs, washed with NaHCO₃ (aq), extracted with CH₂Cl₂ (2×10 ml) and dried over MgSO₄ and filtered. Evaporation of the solvent furnished a white solid, FFR-ck ah (40 mg).

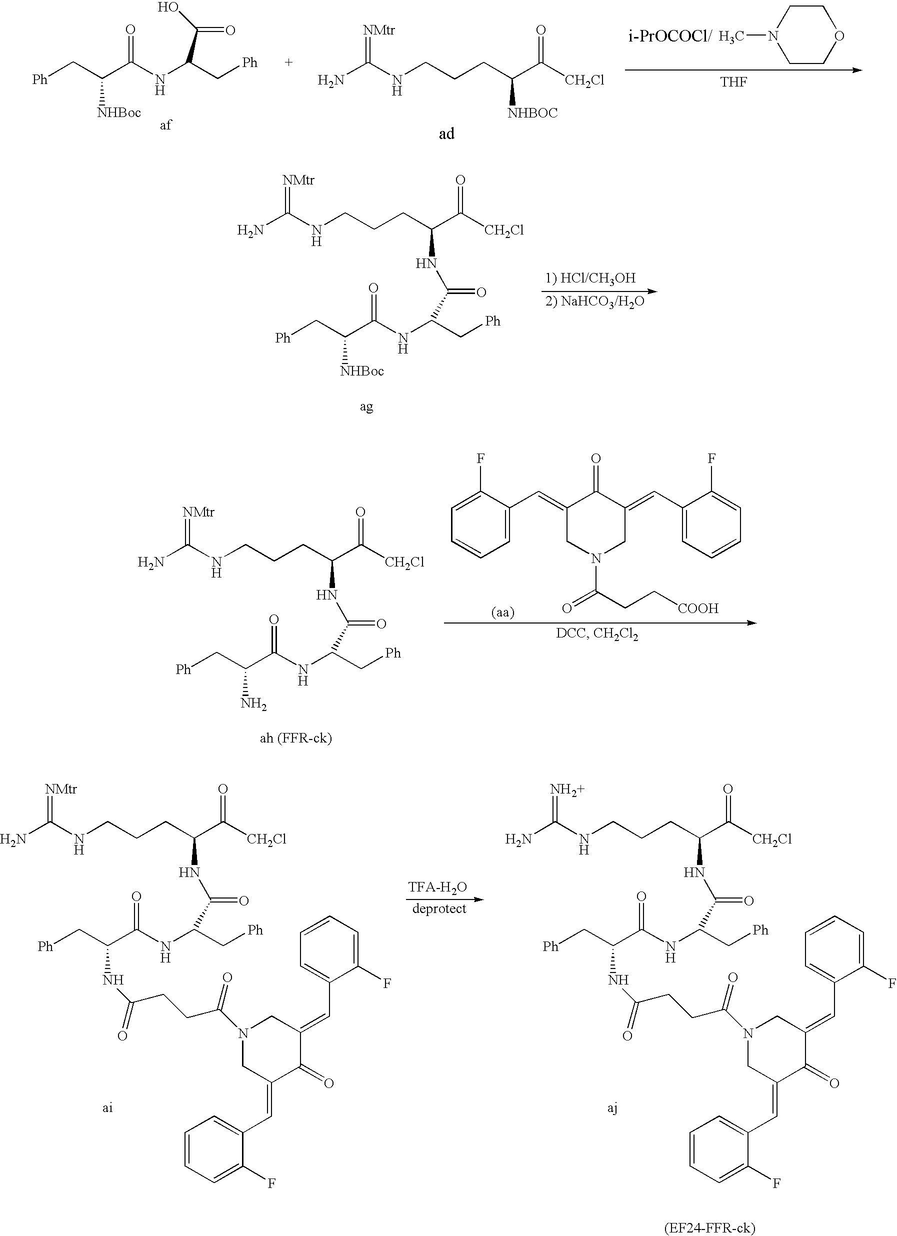

To a mixture of ah (24 mg, 0.032 mmol) and aa (12 mg, 0.03 mmol) in CH₂Cl₂ (0.6 ml) was added DCC (6.18 mg, 0.03 mmol). After stirring overnight at room temperature, filtration and evaporation of the solvent, and purification by flash chromatography using ethyl acetate as the eluant ai (17 mg, 49% yield) was obtained. Compound ai (34 mg, 0.03 mmol) was dissolved in 95% aqueous TFA (0.95 ml) with thioanisole (0.05 ml). The resulting dark solution was stirred for 48 hrs at room temperature and then concentrated under vacuum. The resulting solid was triturated with ether, recrystallized and dried under a vacuum to supply compound aj (EF24-FFR-ck) (12 mg, 45% yield).

### EXAMPLE 3

### Coupling of EF24-FFRck (aj) and fVIIa

Method 1: Recombinant fVIIa (250 μg) was resuspended in 0.5 ml of distilled water and dialyzed in 1 liter of 1 mM TrisHCI, pH 8.0 at 4° C. overnight. Forty-fold molar excess of EF24-FFRck aj synthesized as described in Example 2 above, in 0.25 ml of DMSO was added to a final concentration of 400 μM. The mixture was covered with aluminum foil (EF24 is photosensitive) and incubated at room temperature overnight in darkness. The reaction mixture was centrifuged at 16,000 rpm at 4° C. for 20 minutes in a Sorvall centrifuge to precipitate unbound EF24-FFRck and separate it from EF24-FFRck-fVIIa. The supernatant was further dialyzed in 100 ml of sterile cell culture medium containing 10% fetal bovine serum, penicillin (100 units/ml) and streptomycin 100 μg/ml) at 4° C. overnight. EF24-FFRck-fVIIa equilibrated with the culture medium was added to cells in wells of a 96-well plate.
Method 2: (1). Factor VIIa (fVIIa) will be dialyzed against 1.0 mM Tris HCI, pH 7.4 at 4° C. overnight. (2) EF24-FFRck will be dissolved in 100% DMSO. (3) fVIIa per ml and EF24-FFRck per 0.25 ml will be mixed at a molar ratio 1:13.2 and gently stirred for 2 hrs at room temperature. (4) an additional EF24-FFRck per 0.25 ml (at a molar ratio of 1:13.2) will be added to the reaction mixture to make the final molar ratio 1:40 and continue the coupling reaction overnight at room temperature. (5) the coupled product EF24-FER-ck-fVIIa will be separated from uncoupled EF24-FFRck by column chromatography and 0.5 ml fractions collected. (6) a protein peak (fVIIa) will be determined by reading fractions at OD₂₈₀ and the Bradford protein determination (Bio-Rad) (7) active fractions will be pooled.

### EXAMPLE 4

### Mass Spectroscopic Examination of the Coupled EF24-FFRck (al) to fVIIa

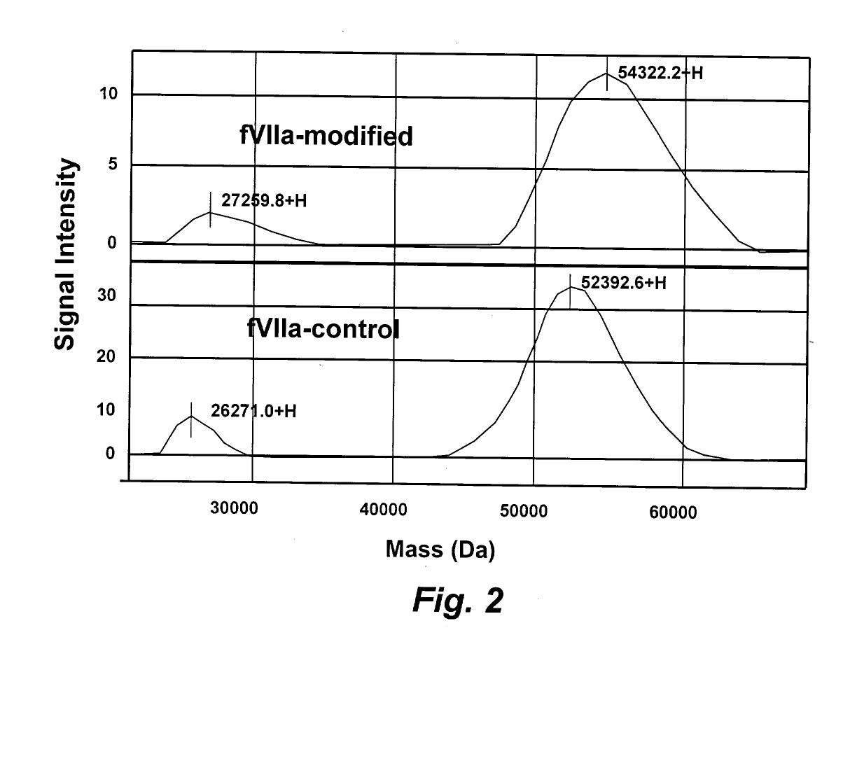

Mass for fVIIa_ is 52392.6+H Daltons, and for EF24-FFRck-fVIIa is 54322.2+H Daltons. The mass of the latter is 1929.6 Dalton greater than the former. MW of EF24-FFRck (894)−HCI (37)=857. 1929.6 divided by 857=2.3. At least 2 molecules of EF24-FFRck were covalently attached to fVIIa, as shown in FIG. 2.

### EXAMPLE 5

### EF24-FFRck-fVIIa Binds Only TF via fVIIa and Kills Human Prostate Cancer Cell Lines

Tissue factor (TF) and vascular endothelial growth factor (VEGF) levels expressed by DU145 and PC3 prostate cancer cell lines were measured by ELISA, as shown in Table 1 below. High TF and VEGF levels were found in DU145 cells.

TABLE 1
Tissue factor (TF) and vascular endothelial growth factor (VEGF)
ELISA in DU145 and PC3 prostate cancer cell lines. High TF and VEGF
levels in DU145 cells. Values indicate Mean ± S.D.

| | |
|---|---|
| TF (pg/ml) | VEGF (pg/ml) |

| DU-145 | PC-3 | DU-145 | PC-3 |
|---|---|---|---|
| 10690 ± 650 | 230 ± 16 | 30511 ± 5748 | 2186 ± 307 |

DU145 cells were plated with 2×10⁴ cells/100 μl/well in a 96 well plate and cultured overnight. The cells were cultured for 48 hrs. Cultures was terminated by adding 40% TCA to a final concentration of 10%. Cells were fixed in TCA at 4° for 1 hr, washed with tap water 5 times and air dried. Sulforhodamine B (SRB) solution (100 μl) at 0.4% (w/v) in 1% acetic acid was added to each well, and the cells were incubated for 10 mins at room temperature. After staining, unbound dye was removed by washing five times with 1% acetic acid and air dried. Bound dye was subsequently solubilized with 200 μl of 10 mM Trizma base, and the absorbance was read on an automated plate reader at a wavelength of 490 nm. Assays were performed in triplicate or quadruplicate. An asterisk (*) indicates p<0.0001 by the Student t-test (two-tailed probability). The concentration of EF24-FFRck-fVIIa was estimated based on protein concentration.

EF24-FFRck alone does not kill any cells since it cannot bind the cell surface, as shown in Table 2.

TABLE 2
EF24-FFRck-fVIIa kills DU145, a Human Prostate Cancer Cell Line
which expresses Tissue Factor. SRB Viability Test. Values are Mean S.D.

| | O.D. 570 nm |
|---|---|
| Control (0.5% DMSO) | 0.370 ± 0.015 |
| EF24-FFRck-fVIIa, 0.8 pM | 0.333 ± 0.053 |
| EF24-FFRck-fVIIa, 8 pM | 0.111 ± 0.004* |
| EF24, 0.8 pM | 0.391 ± 0.041 |
| EF24, 8 pM | 0.053 ± 0.025* |
| EF24-FFRck, 0.8 pM | 0.389 ± 0.021 |
| EF24-FFRck, 8 pM | 0.383 ± 0.027 |

### EXAMPLE 6

### EF24-FFRck-fVIIa Kills Human Breast Cancer and Melanoma Cells

TABLE 3
EF24-FFRck-fVIIa kills Human Breast Cancer (MDA-MB-231) and
Melanoma (RPMI-7951) NR Viability Test. Values indicate Mean ± S.E.

| | O.D. 570 nm | |
|---|---|---|
| | MDA231 | RPM17951 |
| Control (0.5% DMSO) | 0.193 ± 0.019 | 0.269 ± 0.019 |
| EF24-FFRck-fVIIa, 0.5 | 0.142 ± 0.010 | 0.292 ± 0.028 |
| pM | | |
| EF24-FFRck-fVIIa, 2 pM | 0.041 ± 0.002* | 0.066 ± 0.002* |
| EF24, 1 pM | 0.172 ± 0.020 | 0.220 ± 0.023 |
| EF24, 2 pM | 0.109 ± 0.014* | 0.119 ± 0.018 |
| EF24-FFRck, 1 pM | 0.191 ± 0.013 | 0.253 ± 0.018 |
| EF24-FFRck, 2 pM | 0.171 ± 0.009 | 0.247 ± 0.020 |

Student t-test (two-tailed probability)(95% confident level)

The Neutral Red (NR) dye viability assay, instead of the Sulforhodamine B (SRB) assay, was used. In the NR viability assay, NR dye is taken up only by viable cells, while in the SRB viability assay, viable cells are fixed by trichloracetic acid (TCA) on the plate (thus, cells are killed), and the fixed cells are stained by SRB dye.

At the termination of culture, medium was removed and 200 μl of fresh, warm medium containing 50 μg of NR/ml was added to each well in a 96-well plate. Cells were incubated at 37° for 30 mins, followed by two washes with 200 μl of PBS. The NR taken up by cells was dissolved by adding 200 μl of 0.5N HCI containing 35% ethanol. The amount of the dye in each well was read at 570 nm by an ELISA plate reader.

### EXAMPLE 7

### EF24-FFRck-fVIIa Has No Effect on Normal Human Melanocytes and Normal Human Breast Luminal Ductal Cells

TABLE 4
EF24-FFRck-fVIIa has no effect on normal human melanocytes and
MCF10 (normal human breast luminal ductal cell line) which do not
express Tissue Factor: NR (neutral red dye) Viability Test. Value
are Mean ± S.D.

O.D 570 nm

| | Melanocytes | Normal Breast Cells |
|---|---|---|
| Control (None) | 0.264 ± 0.023 | 0.106 ± 0.006 |
| DMSO (1%) | 0.261 ± 0.012 | 0.107 ± 0.012 |
| EF24-FFRck-fVIIa, 4 pM | 0.210 ± 0.005 | 0.096 ± 0.023 |
| EF24, 0.8 pM | 0.255 ± 0.009 | 0.104 ± 0.018 |
| EF24, 4 pM | 0.119 ± 0.009* | 0.091 ± 0.007** |
| EF24-FFRck, 0.8 pM | 0.252 ± 0.007 | 0.101 ± 0.013 |
| EF24-FFRck, 4 pM | 0.249 ± 0.015 | 0.113 ± 0.003 |

*p = 0.002,
**p = 0.031
Assays were performed essentially the same as for DU145 above.

### EXAMPLE 8

### EF24-FFRck-fVIIa Does Not Kill Normal HUVECs

TABLE 5
EF24-FFRck-fVIIa does not kill normal HUVECs that do not express
tissue factor: SRB Viability Test (NCI method). Mean ± S.D.

| | O.D. 490 nm |
|---|---|
| Control (0.5% DMSO) | 0.119 ± 0.003 |
| EF24-FFRck-fVIIa, 0.8 pM | not done |
| EF24-FFRck-fVIIa, 8 pM | 0.370 ± 0.027^{a} |
| EF24, 0.8 pM | 0.136 ± 0.010 |
| EF24, 8 pM | 0.038 ± 0.010* |
| EF24-FFRck, 0.8 pM | 0.152 ± 0.026 |
| EF24-FFRck, 8 pM | 0.160 ± 0.038 |

*Student t-test (two-tailed probability)(95% confident level)
^{a}Cells were not washed before adding the SRB dye and therefore precipitated EF24-FFRck-fVIIa adsorbed dye thereby giving a false elevated .D. 490 nm value
EF24-FFRck-fVIIa does not kill normal HUVECs that do not express surface bound tissue factor.

### EXAMPLE 9

### EF24-FFRck-fVIIa Kills HUVECs Induced to Express TF by 100 nM TPA

TABLE 6
EF24-FFRck-fVIIa kills HUVECs induced to express TF by 100 nM TPA
(phorbol ester) for 24 hrs prior to adding EF24-FFRck-fVIIa: SRB
Viability Test (NCI method). Mean ± S.D.

| | TPA | O.D. 490 nm |
|---|---|---|
| EF24-FFRck-fVIIa, 0.6 pM | 0 | 0.170 ± 0.015 |
| EF24-FFRck-fVIIa, 0.6 pM | + | 0.059 ± 0.004* |

*Student t-test (two-tailed probability)(95% confident level)

### EXAMPLE 10

### Novel Curcumin Analogs (A279L. A279U and EF-15) Are Not Cytotoxic to Vascular Endothelial Cells.

HUVECs, MS-1 cells and SVR cells were cultured to confluence and agents were incubated for 24 hrs. Cell viability was determined by Neutral Red assays. Among synthetic curcumin analogs, A279L, A279U and EF-15 were not cytotoxic at 20 μM. MS-I cells were murine vascular endothelial cells which were immortalized by transfection of SV40 large T antigen but are non-maligant. However, when MS-1 cells were transfected with a ras mutant gene, cells were transformed to become malignant angiosarcoma cells, (SVR cells).

TABLE 7
Novel curcumin analogs (A279L. A279U and EF-15) are not cytotoxic
to vascular endothelial cells.

| | Neutral Red Viability Assay (% of Control) | | |
|---|---|---|---|
| | HUVECs | MS-1 Cells | SVR cells |
| DMSO | 100 | 100 | 100 |
| (0.1%)(control) | | | |
| Curcumin (1 μM) | 103 | 90 | 88 |
| (20 μM) | 21 | 3 | 4 |
| A279L (20 μM) | 97 | 90 | 90 |
| A279U (20 μM) | 92 | 96 | 92 |
| EF-15 (20 μM) | 100 | 95 | 100 |
| C.V.6 (1 μM) | 60 | 33 | 62 |
| (10 μM) | 26 | 10 | 3 |
| C.V.10 (1 μM) | 100 | 68 | 75 |
| (10 μM) | 17 | 7 | 5 |
| EF-2 (1 μM) | 24 | 8 | 20 |
| (10 μM) | 9 | 3 | 2 |
| EF-4 (1 μM) | 14 | 4 | 7 |
| (10 μM) | 15 | 4 | 6 |
| EF-17 (1 μM) | 93 | 87 | 75 |
| (10 μM) | 47 | 10 | 12 |
| EF-25 (1 μM) | 17 | 6 | 28 |
| (10 μM) | 17 | 6 | 4 |
| A283 (1 μM) | 38 | 21 | 37 |
| (10 μM) | 17 | 4 | 3 |
| A286 (1 μM) | 30 | 11 | 24 |
| (10 μM) | 17 | 8 | 4 |
| A287 (1 μM) | 80 | 43 | 67 |
| (10 μM) | 15 | 6 | 4 |

### EXAMPLE 11

### Internalization of TF/FFR-ck-VIIa Complexes After Incubating Cells with Varying Concentrations of FFR-ck-fVIIa for 24 hrs

In three human cancer cell lines (high TF and VEGF producers), FFR-ck-VIIa alone caused internalization of TF into caveolac in the plasmalemma vesicles (Triton X-100 insoluble region of cell membrane) in a dose-dependent manner. FFR-ck-VIIa totally inhibited TF, which remained on the cell surface, to catalyze factor X to generate factor Xa. However, VEGF production and cell viability were not affected. In MDA-MB-231 cells, approximately 10 μM of FFR-ck-VIIa will be required to internalize 50% of TF-FFR-ck-VIIa complexes because MDA-MB-231 human breast cancer cells express greater level of TF than other cell lines.

TABLE 8
Effect of FFRck-fVIIa on cancer cells.

| | TF (nM) on the cell surface | | |
|---|---|---|---|
| | FFR-ck-VIIa (nM) | | |
| Tumor Cell Line | 0 | 100 | 1000 |
| Hs294T | 6.0 ± 0.7 | 3.9 ± 0.6* | 2.5 ± 0.3* |
| RPMI7951 | 81.6 ± 4.5 | 38.7 ± 1.4* | 35.0 ± 6.2* |
| MDA-MB-231 | 624.8 ± 42.0 | 465.5 ± 17.7* | 488.9 ± 1.6* |
| | Percentage relative to 0 nM FFR-ck-VIIa control | | |
| Hs294T | 100 | 65* | 42* |
| RPMI7951 | 100 | 76* | 48* |
| MDA-MB-231 | 100 | 91* | 80* |
| | Percentage internalized relative to 0 nM | | |
| | FFR-ck-VIIa control | | |
| Hs294T | 0 | 35 | 58 |
| RPMI7951 | 0 | 24 | 52 |
| MDA-MB-231 | 0 | 9 | 20 |

Values of TF indicate mean ± S.D. of triplicate determinations.
*Statistically significantly different from control values (p < 0.05).

### EXAMPLE 12

### Dissociation of Chemical Linkage Between Curcumin or Its Analogs and FFR-ck-VIIa (or YGR-ck-VIIa) Inside the Cells

1. Physical Analysis by HPLC Chromatography: Coupled compound such as EF24-FFR-ck-VIIa will be added to a confluent monolayer of cancer cells at an appropriate concentration and incubated for about 2-6 hours. Supernatants will be stored at −20° C. for VEGF ELISA assay. To dissociate surface-bound analog-FFR-ck-VIIa from TF, cells will be harvested with a rubber policeman and resuspended in 200 μl of ice-cold phosphate buffered saline (PBS)/HCl (pH 3.0) for 1 min at 0° C. The cells will be spun for 5 secs in a microfuge centrifuge and supernatants removed. Cell viability will not affected by exposure to acid. To the cell pellet, 0.5 ml of ice-cold 10 mM Tris/HCl (pH 7.4) will be added and sonicated for 10-20 secs. and solubilized with 1% Triton X-100 overnight. Cells will then pelleted by centrifugation. Proteins in the supernatants of the extracts will be measured by the Bradford method (Bio-Rad). The aliquot of the solubilized extract from each sample containing an equal amount of total protein will be passed through a membrane filter with a pore size 1,000-2,000 to separate analogs from larger proteins. The filtered extract containing analogs will be chromatographed by HPLC. Another aliquot will be used for quantifying TF by ELISA. The presence of a single peak of the analog separated from the FFR-ck-VIIa, TF, FFR-ck-VIIa-TF, or analog-FFR-ck-VIIa-TF peaks will be taken as evidence of dissociation.

FFR-ck-VIIa as a negative control and the analog alone as a positive control will be added to the monolayers, cultured for 6 hours and the solubilized fraction will be similarly analyzed. HPLC will be performed using a Beckman liquid chromatograph equipped with a pump, a UV/vis. detector and a recorder. A Waters Nova-Pak C₁₈ column (150×3.9 mm, 5-μm particle size) will be used. The mobile phase will consist of 40% tetrahydrofuran and 60% water containing 1% citric acid, adjusted to pH 3.0 with concentrated KOH solution (v/v). The system will be run isocratically at a flow rate of 1 ml/min. Sample detection will be achieved at 420 nm, and injection volumes will be 20 μl. Calibration curves over the range of 0.2 to 20 μM will be established for the quantitation of curcumin analogs. This HPLC detection method will offer a detection limit of 5 ng/ml.

### EXAMPLE 13

### Functional Analysis by TF and VEGF Production and Neutral Red (NR) Viability Assay

TF and VEGF levels were quantified by ELISA in the samples obtained by experiment as described above and adjusted based on protein concentration of the samples. In addition, cancer cells were grown to confluency in 48-well plates in duplicate. Each well was incubated with analog-FFR-ck-VIIa, analog alone, FFR-ck-VIIa alone, or DMSO (solvent control) for 4 days. Supernatants were collected for qualifying VEGF levels by ELISA. One plate was used to determine cell viability by NR assay. The other plate was used to determine levels of TF (in the cells) by ELISA. Levels of VEGF and TF in each well were adjusted by the value of neutral red assay.

### EXAMPLE 14

### Curcuminoid EF24 Is More Effective than Curcumin Against Tumor Cells

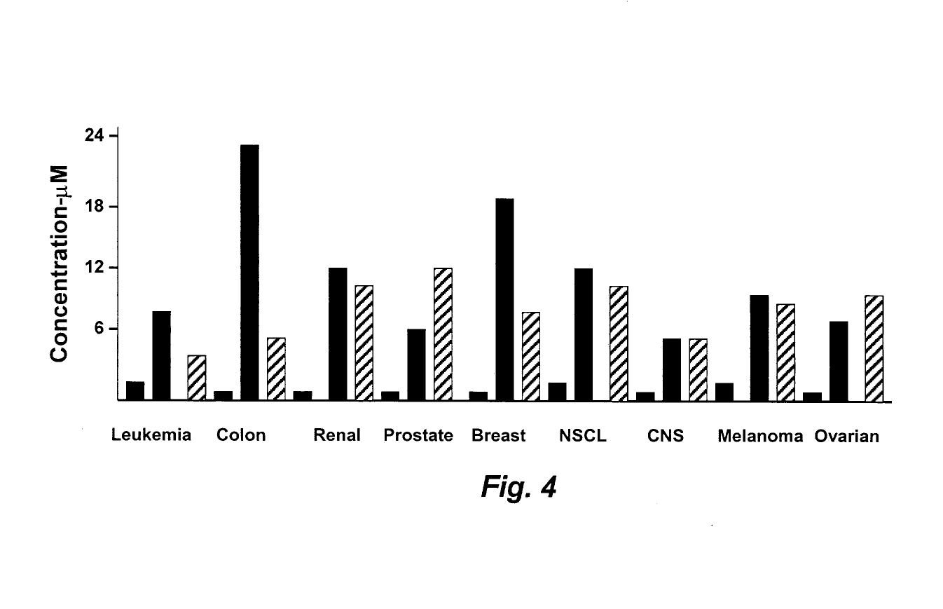

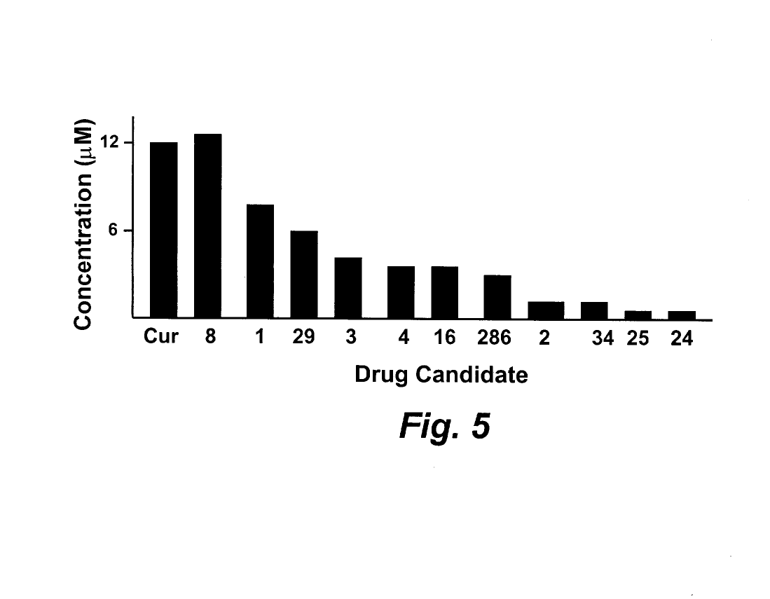

Curcumin, EF24 and cisplatin were tested against tumor cells in the NCI screening system. EF24 was significantly more effective than either cisplatin or curcumin, as shown in FIG. 4. Curcuminoids were also added to transformed breast cancer cells and the mean growth inhibitory concentrations determined, as shown in FIG. 5.

What is claimed is:

1. A composition comprising:
(a) a protein, wherein the protein selectively binds a surface marker of a target cell;
(b) at least one linker covalently bonded to the protein; and
(c) a cytotoxic compound bonded to the linker by a hydrolysable bond.

2. The composition according to claim 1, wherein the protein selectively binds to tissue factor on the surface of the target cell.

3. The composition according to claim 1, wherein the protein is a component polypeptide of a factor VIIa.

4. The composition according to claim 1, wherein the protein is a component polypeptide of a factor VIIa, and wherein the polypeptide comprises the amino acid sequence between amino acid positions 153 and 406 of SEQ ID NO: 1 or a truncated or modified variant thereof

5. The composition according to claim 1, wherein the protein is selected from an antibody and tissue factor pathway inhibitor.

6. The composition according to claim 1, wherein the protein is capable of being internalized by the target cell.

7. The composition according to claim 1, wherein the at least one linker is a peptidyl linker.

8. The composition according to claim 7, wherein the at least one peptidyl linker is a peptidyl methylketone linker.

9. The composition according to claim 1, wherein the composition further comprises a tether.

10. The composition according to claim 1, wherein the at least one linker is a tether.

11. The composition according to claim 1, wherein the hydrolysable bond is selected from the group consisting of a carbamate, an amide, an ester, a carbonate and a sulfonate.

12. The composition according to claim 1, wherein the at least one linker is an arginyl methylketone selected from the group consisting of phenylalanine-phenylalanine-arginine methylketone, tyrosine-glycine-arginine methylketone, glutamine-glycine-arginine methylketone, glutamate-glycine-arginine methylketone and phenylalanine-proline-arginine methylketone.

13. The composition according to claim 1, wherein the at least one linker is selected from tyrosine-glycine-arginine methylketone and phenylalanine-phenylalanine-arginine methylketone.

14. The composition according to claim 1, wherein the at least one linker is phenylalanine-phenylalanine-arginine methylketone.

15. The composition according to claim 1, wherein the at least one linker is tyrosine-glycine-arginine methylketone.

16. The composition according to claim 3, wherein at least one linker is covalently bonded to an amino acid side chain within a serine protease active site of factor VIIa, thereby inactivating the serine protease active site.

17. The composition according to claim 1, wherein the cytotoxic compound is a curcuminoid having the formula: 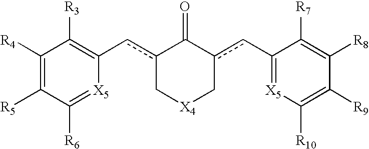
wherein:
X₄ is (CH₂)ₘ, O, S, SO, SO₂, or NR₁₂, where R₁₂ is H, alkyl, substituted alkyl, acyl, alkoxycarbonyl, aminocarbonyl, alkylaminocarbonyl or dialkylaminocarbonyl;
m is 1-7;
each X₅ is independently N or C—R₁₁;
and each R₃-R₁₁ are independently H, halogen, hydroxyl, alkoxy, CF₃, alkyl, substituted alkyl, alkenyl, alkynyl, cycloalkyl, substituted cycloalkyl, aryl, substituted aryl, alkaryl, arylalkyl, heteroaryl, substituted heteroaryl, heterocycle, substituted heterocycle, amino, alkylamino, dialkylamino, carboxylic acid, carboxylic ester, carboxamide, nitro, cyano, azide. alkylcarbonyl, acyl, or trialkylammonium; and the dashed lines indicate optional double bonds; with the proviso that when X₄ is (CH₂)ₘ, m is 2-6, and each X₅ is C—R₁₁, R₃-R₁₁ are not alkoxy, and when X₄ is NR₁₂ and each X₅ is N, R₃-R₁₀ are not alkoxy, alkyl, substituted alkyl, alkenyl, alkynyl, cycloalkyl, substituted cycloalkyl, aryl, substituted aryl, alkaryl, arylalkyl, heteroaryl, substituted heteroaryl, amino, alkylamino, dialkylamino, carboxylic acid, or alkylcarbonyl, and wherein the stereoisomeric configurations include enantiomers and diastereoisomers, and geometric (cis-trans) isomers.

18. The composition according to claim 13, wherein X₄ is selected from the group consisting of —NH and —NR₁₂.

19. The composition according to claim 13, wherein R₃-R₁₀ is selected from hydroxyl and —NHR₁₂.

20. The composition according to claim 1, wherein the cytotoxic compound is a curcuminoid having the formula:

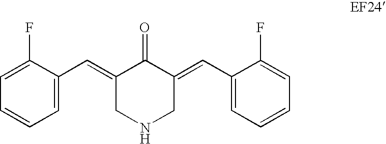

21. The composition according to claim 1, wherein the tether is selected from the group consisting of a dicarboxylic acid, a disulfonic acid, an omega-amino carboxylic acid, an omega-amino sulfonic acid, an omega-amino carboxysulfonic acid, or a derivative thereof, wherein the tether comprises 2-6 carbons, and wherein the tether is capable of forming a hydrolysable bond.

22. The composition according to claim 1, wherein the tether comprises a dicarboxylic acid.

23. The composition according to claim 1, wherein the tether is succinate.

24. A pharmaceutical composition comprising a protein, wherein the protein selectively binds a surface marker of a target cell, and wherein the protein is covalently bonded to at least one linker, wherein each linker has a cytotoxic compound bonded thereto, and wherein said cytotoxic compound is covalently linked by hydrolysable bond to the linker, and a pharmaceutically acceptable carrier.

25. The pharmaceutical composition of claim 24 further comprising a tether covalently linked by hydrolysable bond to the cytotoxic compound.

26. The pharmaceutical composition according to claim 24, wherein the hydrolysable bond is selected from the group consisting of a carbamate, an amide, an ester, a carbonate and a sulfonate.

27. The pharmaceutical composition according to claim 25, wherein the tether is selected from the group consisting of a dicarboxylic acid, a disulfonic acid, an omega-amino carboxylic acid, an omega-amino sulfonic acid, an omega-amino carboxysulfonic acid, or a derivative thereof, wherein the tether comprises 2-6 carbons, and wherein the tether is capable of forming a hydrolysable bond.

28. The pharmaceutical composition according to claim 24, wherein the at least one linker is an arginyl methylketone selected from the group consisting of phenylalanine-phenylalanine-arginine methylketone, tyrosine-glycine-arginine methylketone, glutamine-glycine-arginine methylketone, glutamate-glycine-arginine methylketone and phenylalanine-proline-arginine methylketone.

29. The pharmaceutical composition of claim 24, wherein the cytotoxic compound is a curcuminoid having the formula:

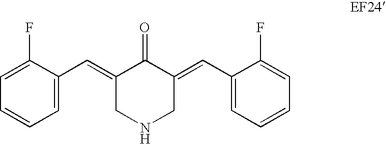

30. The pharmaceutical composition of claim 24, formulated in a pharmaceutically effective dosage amount.

31. The pharmaceutical composition of claim 24, wherein the protein is a component polypeptide of a factor VIIa.

32. The pharmaceutical composition of claim 24, wherein the pharmaceutical composition is formulated for intravenous infusion.

33. A method of producing a cytotoxic compound-protein conjugate, comprising the steps of:
(a) synthesizing a product comprising a cytotoxic compound;
(b) bonding covalently the product of step (a) and the linker; and
(c) covalently bonding at least one molecule of the composition of step (b) to a protein capable of selectively binding to a surface marker of a target cell.

34. The method of claim 33, wherein the cytotoxic compound is a curcuminoid having the formula: 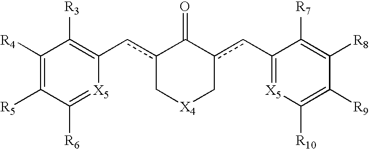
wherein:
X₄ is (CH₂)ₘ, O, S, SO, SO₂, or NR₁₂, where R₁₂ is H, alkyl, substituted alkyl, acyl, alkoxycarbonyl, aminocarbonyl, alkylaminocarbonyl or dialkylaminocarbonyl;
m is 1-7;
each X₅ is independently N or C—R₁₁;
and each R₃-R₁₁ are independently H, halogen, hydroxyl, alkoxy, CF₃, alkyl, substituted alkyl, alkenyl, alkynyl, cycloalkyl, substituted cycloalkyl, aryl, substituted aryl, alkaryl, arylalkyl, heteroaryl, substituted heteroaryl, heterocycle, substituted heterocycle, amino, alkylamino, dialkylamino, carboxylic acid, carboxylic ester, carboxamide, nitro, cyano, azide. alkylcarbonyl, acyl, or trialkylammonium; and the dashed lines indicate optional double bonds; with the proviso that when X₄ is (CH₂)ₘ, m is 2-6, and each X₅ is C—R₁₁, R₃-R₁₁ are not alkoxy, and when X₄ is NR₁₂ and each X₅ is N, R₃-R₁₀ are not alkoxy, alkyl, substituted alkyl, alkenyl, alkynyl, cycloalkyl, substituted cycloalkyl, aryl, substituted aryl, alkaryl, arylalkyl, heteroaryl, substituted heteroaryl, amino, alkylamino, dialkylamino, carboxylic acid, or alkylcarbonyl, and wherein the stereoisomeric configurations include enantiomers and diastereoisomers, and geometric (cis-trans) isomers.

35. The method of claim 33, wherein step (a) comprises reacting the curcuminoid with a tether selected from the group consisting of a dicarboxylic acid, a disulfonic acid, an omega-amino carboxylic acid, an omega-amino sulfonic acid, an omega-amino carboxysulfonic acid, or a derivative thereof, wherein the tether comprises 2-6 carbons, and wherein the tether is capable of forming a hydrolysable bond.

36. The method of claim 34, wherein X₄ is selected from the group consisting of —NH and —NR₁₂.

37. The method of claim 34, wherein R₃-R₁₀ is selected from hydroxyl and —NHR₁₂.

38. The method of claim 33, wherein the cytotoxic compound has the formula:

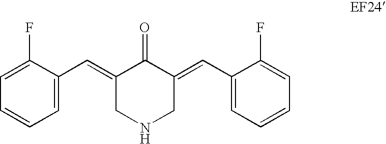

39. The method of claim 33, wherein step (a) comprises reacting the cytotoxic compound with a dicarboxylic anhydride.

40. The method of claim 39, wherein the dicarboxylic anhydride is succinic anhydride.

41. The method of claim 39, wherein the product of step (a) has the formula:

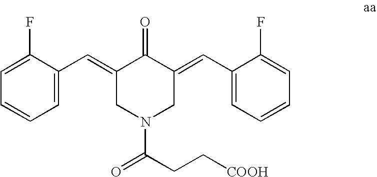

42. The method of claim 33, wherein the step (b) comprises the step of providing a peptidyl linker.

43. The method of claim 42, wherein the step (b) comprises the steps of:
(i) reacting a composition having the formula: 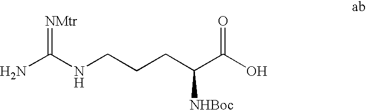
with isopropyl chloroformate and ethereal diazomethane, thereby producing a compound having the formula: 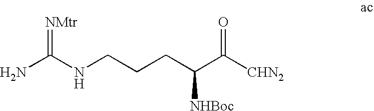
(ii) reacting a compound having the formula: 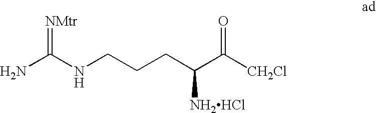
with N-Boc-Phe-Phe-OH, isopropyl chloroformate, and a base; thereby producing a compound having the formula: 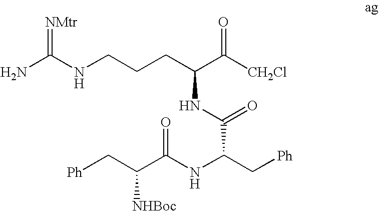
(iii) deprotecting compound ag, thereby producing a compound having the formula:

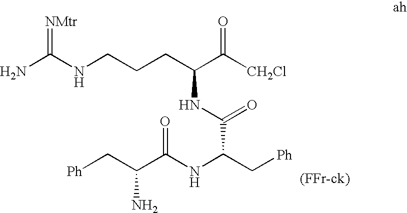

44. The method of claim 33, wherein the composition of step (b) has the formula:

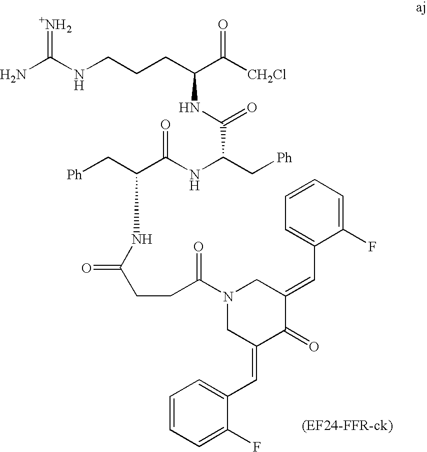

45. The method of claim 33, wherein the protein is a component polypeptide of a factor VIIa.

46. The method of claim 33, wherein at least one molecule of the composition of step (b) is covalently bonded to an amino acid of the serine protease active site of factor VIIa, thereby inactivating the active site.

47. The method of claim 36, wherein the amino acid is the His193 of SEQ ID NO: 1.

48. A method of modulating a physiological function of a target cell, comprising the steps of contacting a target cell having a surface marker thereon with a composition according to claim 1, whereby the composition selectively binds to the surface marker and is internalized, thereby releasing the cytotoxic compound from the protein; and modulating the physiological function of the target cell.

49. The method according to claim 48, wherein the surface marker is tissue factor.

50. The method according to claim 48, wherein the physiological function is proliferation of the cell, and wherein proliferation is reduced.

51. The method according to claim 48, wherein the target cell is selected from a vascular endothelial cell, a vascular smooth muscle cell, a tumor cell, a monocyte, a macrophage and a microparticle.

52. The method according to claim 48, wherein the target cell is a vascular endothelial cell.

53. The method according to claim 48, wherein the target cell is a vascular smooth muscle cell.

54. The method according to claim 48, wherein the vascular endothelial cell is selected from the group consisting of an isolated vascular endothelial cell, a capillary endothelial cell, a venal endothelial cell, an arterial endothelial cell and a neovascular endothelial cell of a tumor.

55. The method according to claim 48, wherein the composition further comprises a pharmaceutically acceptable carrier.

56. The method according to claim 48, wherein the target cell is an cultured cell.

57. The method according to claim 48, further comprising the step of delivering the composition to an animal or human having the target cell.

58. The method according to claim 48, wherein the composition is delivered to an animal or human by a route selected from the group consisting of topical intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, intrasternal injection and infusion.

59. A method of selectively delivering a cytotoxic compound to a target cell, comprising the steps of:
(a) contacting a target cell having a surface marker thereon with a composition according to claim 1; and
(b) binding the composition to the surface marker on the target cell, whereby the composition is internalized by the target cell, thereby delivering the cytotoxic compound to the interior of the target cell.

60. The method according to claim 59, wherein the therapeutic preparation further comprises a pharmaceutically acceptable carrier.

61. The method according to claim 59, wherein the cytotoxic compound is a curcuminoid having the formula: 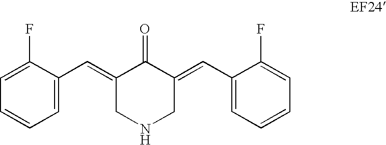
and wherein the protein is a component polypeptide of factor VIIa.

62. A method of modulating a pathological condition in an animal or human, comprising the step of administering to an animal or human subject having a pathological condition an effective dose of a composition according to claim 1, thereby reducing the proliferation of a target cell capable of expressing surface-bound marker, and thereby modulating the pathological condition of the patient subject.

63. The method according to claim 62, wherein the surface marker of the target cell is tissue factor.

64. The method according to claim 62, wherein the pathological condition is selected from the group consisting of cancer, hypercoagulapathy, restenosis, diabetic retinopathy, rheumatoid arthritis and a skin disorder inflammation.

65. The method according to claim 62, wherein the pathological condition is a cancer selected from the group consisting of leukemia, breast cancer, lung cancer, liver cancer, melanoma and prostrate cancer.

66. The method according to claim 62, wherein the target cell is a vascular endothelial cell.

67. The method according to claim 62, wherein the target cell is a vascular smooth mucscle cell.

68. The method according to claim 62, wherein the target cell is a cancer cell.

69. The method according to claim 62, wherein the composition is antiangiogenic and wherein reducing proliferation of a target cell reduces angiogenesis.

70. The method according to claim 69, wherein reducing angiogenesis causes a reduction in a tumor.


SEQUENCE LISTING (1 sequence)

SEQ ID NO: 1 - protein, 406 aa, Homo sapiens [sequence omitted]